(19) United States
(12) Patent Application Publication
(10) Pub. No.: US 2021/0174921 A1
(43) Pub. Date: Jun. 10, 2021

(54) BOT SUPPORT IN TRIAGE GROUP COMMUNICATION

(71) Applicant: Orion Labs, Inc., San Francisco, CA (US)

(72) Inventors: Gregory Albrecht, San Francisco, CA (US); Ellen Juhlin, San Francisco, CA (US)

(21) Appl. No.: 17/052,248

(22) PCT Filed: May 7, 2019
(86) PCT No.: PCT/US19/31045

§ 371 Date: Nov. 2, 2020

Related U.S. Application Data
(60) Provisional application No. 62/667,955, filed on May 7, 2018.
(60) Provisional application No. 62/667,982, filed on May 7, 2018.
(60) Provisional application No. 62/667,990, filed on May 7, 2018.

(51) Int. Cl. G16H 15/00; G10L 15/26; H04W 4/80; G16H 40/20

(52) U.S. Cl. CPC G16H 15/00; G16H 40/20; H04W 4/80; G10L 15/26

(57) ABSTRACT

A group management system or service creates a multiple triage node groups in response to an event notification. Each triage node group includes user nodes and a triage bot node that assists in collecting patient data and possibly other triage data from messages sent by the user nodes. The triage node groups are dynamically modified and managed based on evaluation of the collected patient and other triage data. Collected patient data can be prioritized by a triage management system and, in some implementations, the triage bot nodes to generate patient transportation and/or treatment instructions and the like. Medical facilities can be notified of the number of patients that might require attention and the nature of their injuries, etc. Each user node can be a two-device node (e.g., a voice unit paired with an intermediate communication device) or a stand-alone device and accesses appropriate communication networks and messaging services.

### RELATED APPLICATIONS

This application hereby claims the benefit of and priority to PCT Application No. PCT/US2019/031045, entitled “BOT SUPPORT IN TRIAGE GROUP COMMUNICATION,” filed May 7, 2019, and to U.S. Provisional Patent Application No. 62/667,990, entitled “BOT SUPPORT IN PATIENT CARE GROUP COMMUNICATION,” filed May 7, 2018, which are hereby incorporated herein by reference in their entirety.

### TECHNICAL FIELD

Aspects of the disclosure are related to communications and, in particular, to bot support in various types of group communications.

### TECHNICAL BACKGROUND

Internet bots, also known as web robots, robots or simply bots, are software applications that run automated tasks (e.g., scripts) over the Internet and commonly interact with other users, applications, etc. Bots often perform tasks that are both simple and structurally repetitive (including tasks that human users could not perform), at a much higher rate (e.g., speed and/or frequency) than would be possible for a human alone.

Some bots communicate with other users of Internet-based services (e.g., via Instant Messaging (IM), Internet Relay Chat (IRC), or another web interface such as Facebook Bots and Twitterbots). These so-called “chatterbots” may allow people to ask questions or make statements in plain English, then formulate a proper response or take other action. These bots can often handle many tasks (e.g., reporting weather, zip-code information, sports scores, converting currency or other units, etc.). Other bots are used for entertainment, shopping, travel booking, and the like.

### Overview

Implementations providing bot support in triage group communications include systems, methods, and software to create and dynamically modify and manage one or more triage node groups by utilizing triage bot nodes in the triage node groups. A group management/messaging system or service creates one or more triage node groups in response to an emergency event notification. Each triage node group has one or more user nodes and at least one triage bot node. Each triage user node can be configured to receive patient data (and other triage data in some implementations) and to forward the received patient data to the triage bot node that is part of the triage user node's group. In some implementations the triage bot nodes immediately forward the collected patient data to a triage management system for prioritization while in other implementations the triage bot nodes may perform some or all of the prioritization of patient data collected for a given triage node group. Each triage bot node may then forward its node group's patient data (prioritized or not) to a triage management system that performs prioritization to generate a patient transportation (e.g., evacuation) plan or related instructions, and possibly field treatment instructions for one or more patients based on the collected patient data. The triage management system may also send notifications regarding these types of issues to medical facilities to assist them in planning for patient arrival and treatment and to other parties participating in the triage or emergency response effort.

In some implementations one or more triage node group user nodes is a two-device node that includes an end user device (e.g., a voice unit) paired with an intermediate communication device that can be wirelessly linked to a communication network that also is linked to group management/messaging. In other implementations one or more of the triage node group user nodes is a stand-alone device that can access the appropriate communication network and messaging/management service or system. The triage management system and any group management or messaging service or system can operate separately or can operate as a single system (in one location or in a distributed fashion). Each triage bot node also may be configured to lead a user node user through a pre-scripted protocol, through a prescribed data collection process, and/or through a series of questions that are configured to lead the user through a triage patient examination.

Other implementations providing bot support in emergency services group communications include systems, methods, and software to create and dynamically modify and manage one or more response node groups by utilizing risk identification bot nodes in the response node groups. A group management/messaging system or service creates one or more response node groups in response to an emergency event notification. Each response node group has one or more user nodes and at least one risk identification node, which can be a bot node or another user node. A risk identification bot node evaluates data obtained from user node communications such as voice messages sent to and from user nodes in the response node group. When a risk identification bot node determines that an actual or potential risk is or may be present, a risk assessment node such as a risk assessment bot node is added to the response node group to provide additional information about the identified risk based at least in part on the information obtained from user node messages. A risk assessment bot node may be accessed in or obtained from a bot library or the like and may be configured to be a response node group member.

In some implementations one or more response node group user nodes is a two-device node that includes an end user device (e.g., a voice unit) paired with an intermediate communication device that can be wirelessly linked to a communication network that also is linked to group management/messaging. In other implementations one or more response node group user nodes is a stand-alone device that can access the appropriate communication network and messaging/management service or system.

Still other implementations providing bot support in triage group communications include systems, methods, and software to create and dynamically modify and manage one or more triage node groups by utilizing triage bot nodes in the triage node groups. A group management/messaging system or service creates one or more triage node groups in response to an emergency event notification. Each triage node group has one or more user nodes and at least one triage bot node. Each triage user node can be configured to receive patient data (and other triage data in some implementations) and to forward the received patient data to the triage bot node that is part of the triage user node's group. In some implementations the triage bot nodes immediately forward the collected patient data to a triage management system for prioritization while in other implementations the triage bot nodes may perform some or all of the prioritization of patient data collected for a given triage node group. Each triage bot node may then forward its node group's patient data (prioritized or not) to a triage management system that performs prioritization to generate a patient transportation (e.g., evacuation) plan or related instructions, and possibly field treatment instructions for one or more patients based on the collected patient data. The triage management system may also send notifications regarding these types of issues to medical facilities to assist them in planning for patient arrival and treatment and to other parties participating in the triage or emergency response effort.

In some implementations one or more triage node group user nodes is a two-device node that includes an end user device (e.g., a voice unit) paired with an intermediate communication device that can be wirelessly linked to a communication network that also is linked to group management/messaging. In other implementations one or more of the triage node group user nodes is a stand-alone device that can access the appropriate communication network and messaging/management service or system. The triage management system and any group management or messaging service or system can operate separately or can operate as a single system (in one location or in a distributed fashion). Each triage bot node also may be configured to lead a user node user through a pre-scripted protocol, through a prescribed data collection process, and/or through a series of questions that are configured to lead the user through a triage patient examination.

This overview is provided to introduce a selection of concepts in a simplified form that are further described below in the Technical Disclosure. It may be understood that this overview is not intended to identify key features or essential features of the claimed subject matter, nor is it intended to be used to limit the scope of the claimed subject matter.

### BRIEF DESCRIPTION OF THE DRAWINGS

Many aspects of the disclosure can be better understood with reference to the following drawings. While several implementations are described in connection with these drawings, the disclosure is not limited to the implementations disclosed herein. On the contrary, the intent is to cover all alternatives, modifications, and equivalents.

FIG. 1 illustrates a group messaging system in accordance with embodiments of the present disclosure.

FIG. 2 illustrates non-limiting exemplary systems for communication nodes.

FIG. 3A illustrates a messaging process.

FIG. 3B illustrates a messaging process.

FIG. 4 illustrates a messaging process.

FIG. 5A illustrates communications between an end user device and an intermediate communication device.

FIG. 5B illustrates communications between an end user device and an intermediate communication device.

FIG. 6 illustrates bot support in emergency services group communications.

FIG. 7 illustrates a method for bot support in emergency services group communications.

FIG. 8 illustrates bot support in triage group communications.

FIG. 9 illustrates a method for bot support in triage group communications.

FIG. 10 illustrates bot support in patient care group communications.

FIG. 11 illustrates a method for bot support in patient care group communications.

FIG. 12 illustrates a representative computing device in accordance with embodiments of the present disclosure.

### DETAILED DESCRIPTION

The Figures and description describe various embodiments that may be used to provide bot support to a group management service (also referred to in some implementations as a “group messaging service”) that manages a number of communication nodes in emergency responsibility situations and settings. Some implementations will be explained in the non-limiting exemplary setting of first responders who are responding to an emergency. Many arrangements of messaging systems such as the non-limiting examples shown in the Figures are possible, and the illustrated implementations should be viewed as only exemplary arrangements of many such possible implementations.

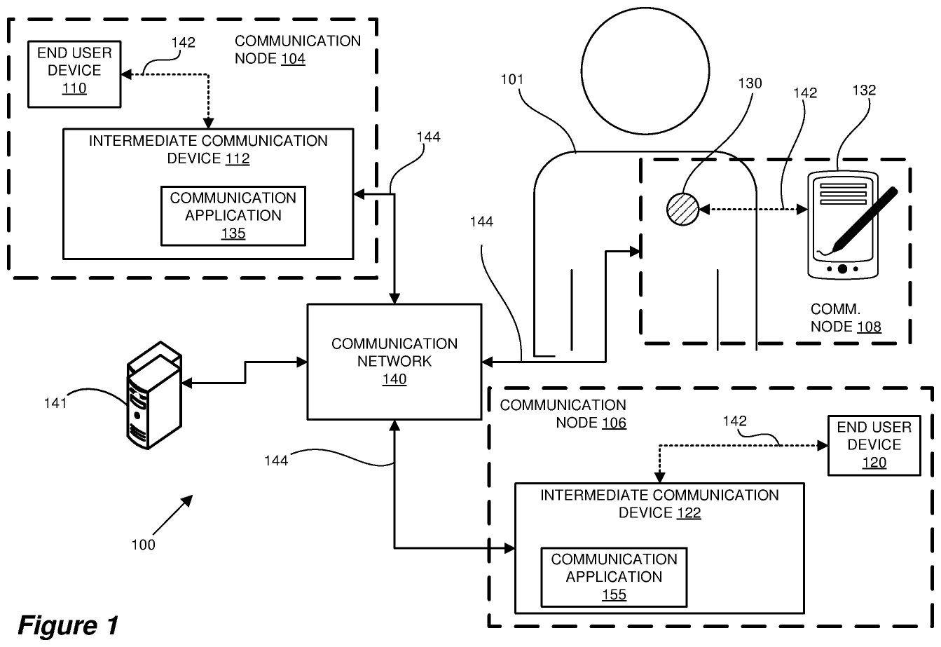

FIG. 1 illustrates one or more implementations of a group messaging system 100 configured to facilitate and manage voice and, in some implementations, other audio communications between endpoints connected to a communications network. System 100 includes communication node 104 (which includes voice unit 110 (also referred to as an end user device) linked to intermediate communication device 112), communication node 106 (which includes voice unit 120 linked to intermediate communication device 122), and communication network 140 linking nodes 104, 106. An additional communication node 108 (associated with a user 101), comprising a voice unit 130 paired with and coupled to an intermediate communication device 132, also is shown connected to network 140. As will be appreciated by those skilled in the art, additional nodes can be interconnected via communication network 140. Other communication devices also might be connected to network 140. The voice units 110, 120, 130 can each be a highly portable, push-to-talk (PTT) voice communication device (e.g., a pendant or other device wearable by a user 101 on clothing or a backpack). Nodes connected to network 140 may be comprised of different types and/or combinations of devices and components, as noted with regard to different node implementations discussed herein. In some implementations a communication node may comprise a voice unit and intermediate communication device linked together; in other implementations, an intermediate communication device executing a group communication application may be considered the communication node; in yet other implementations, an end user device alone may be considered the communication node. Other definitions of a “communication node” are possible as disclosed and/or taught herein.

Intermediate communication device 112 (also referred to as an “ICD”) can be a computing system, non-limiting examples of which include a cellphone, smartphone, gaming device, tablet or laptop. As part of communication node 104, ICD 112 communicates with its linked voice unit 110 over a communication link 142 (various types of links can be used, non-limiting examples of which include Bluetooth and Bluetooth low energy), and further communicates outside node 104 using communication network 140 over one or more communication network links 144. In communication node 106, ICD 122 also communicates with its linked voice unit 120 using a intra-nodal communication link 142, and further communicates outside node 106 using communication network 140 over communication network link 144. In communication node 108, ICD 132 communicates with its linked voice unit 130 using an intra-nodal communication link 142, and further communicates outside node 108 using communication network 140 over communication network link 144.

Intra-nodal links 142 can be used to link an end user device with its associated intermediate communication device using communication linking. The communication links 144 that connect intermediate communication devices 112, 122, 132 to communication network 140 can use one or more known circuit-switched, communication signaling, wired communications, wireless communications, and/or other communication format or protocol, including improvements thereof. Communication links 144 may each be a direct link, or can include intermediate networks, systems (including one or more management systems), or devices, and can include a logical network link transported over multiple physical links. The links 144 and communication network 140 can be the Internet or any part(s) thereof.

Each ICD 112, 122, 132 may comprise a computing system (non-limiting examples of which include a cellphone, smartphone, tablet, gaming device and/or laptop computer) capable of running a communication application and communicating with communications network 140 using the Internet or some other widespread communication network. Each communication node has a node ID that can be used to facilitate group membership and participation. A given communication device (whether an intermediate communication device or other device) communicating with the messaging service may have an IP address that can be used for communications across the Internet and the like, but then may also utilize its node ID for purposes of the node's operating within system 100. The node ID can be a service identity (similar to a Twitter handle or the like) that identifies the user to a communication, messaging and/or other service and/or application (e.g., a cloud-based group messaging service). Moreover, communications network 140 can include management and/or other group communication services (e.g., via a computing system comprising one or more servers 141 or the like), including those described herein. In some implementations the group messaging service is a server-centric, centralized service that manages group communications and other functions as described in connection with one or more implementations discussed herein. Each of ICDs 112, 122, 132 includes at least one user interface that allows a user to enter data and interact with a communication application being executed on the ICD (e.g., applications 135, 155 operating on ICDs 112, 122, respectively). ICD 132 operates similarly, though its communication application is not shown in FIG. 1. When transmitting and receiving data, ICDs 112, 122, 132 and the like can use an appropriate data transfer scheme.

As noted above, communications network 140 can comprise a server system 141 utilizing one or more computing devices capable of providing communication services to a plurality of communication nodes and their respective endpoint devices (e.g., voice units or other end user devices 110, 120, 130). Voice units 110, 120, 130 (also referred to as “VUs”) may each comprise a speaker, microphone, processing system, communication interface, and a user interface to exchange communications with ICDs 112, 122, 132, respectively, and thus with communications network 140 and other users via endpoint devices of various types. In implementations of emergency support bot in group communication service, one or more intermediate communication devices and/or voice units may include modules that are configured to implement functionalities described herein.

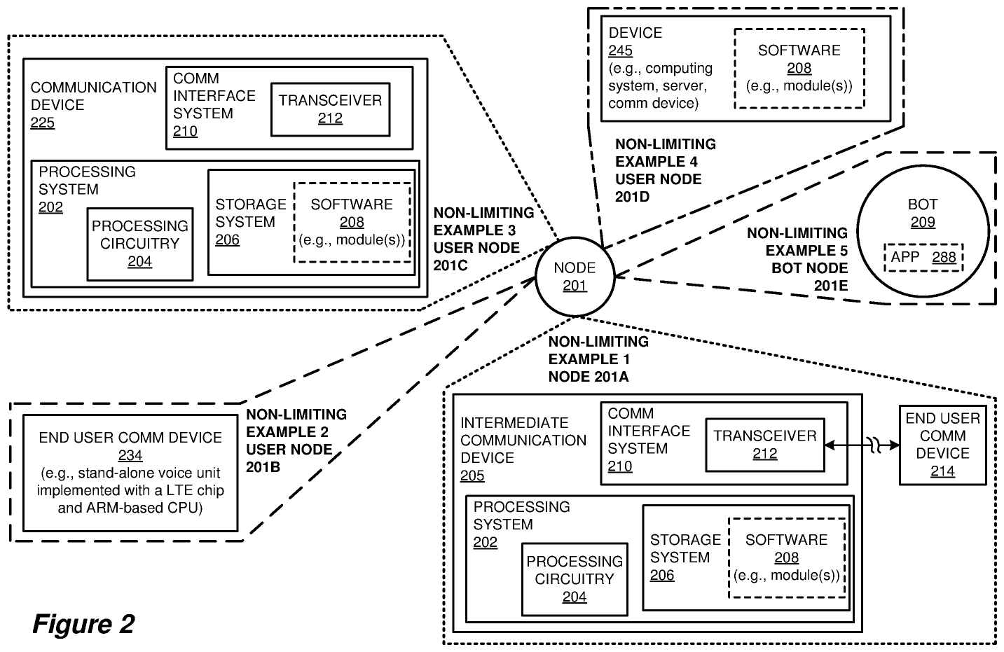

FIG. 2 provides several non-limiting examples of communication nodes 201A, 201B, 201C, 201D that can be included in implementations of the group messaging system of FIG. 1. A node may be one of the following non-limiting exemplary implementations—an end user device by itself, a compound node comprising an end user device plus an intermediate communication device, an intermediate communication device by itself, a software program or application running on a device, or a bot. A node represents whatever entity can send or be sent a message; a node in some implementations can be an addressable entity in a communication group. A communication application being executed on the addressable entity may be the node in such implementations. Because such an application has to run on hardware, the node may further include whatever hardware is used in performing group communication functions described herein. The hardware might be a cellphone or smartphone by itself, or the hardware may include such a cellphone or smartphone linked to a voice unit. In the linked-device implementations, the communication application running on an intermediate communication device may be the node in the network, and it talks to the voice unit via a wireless link (non-limiting examples including Bluetooth or Bluetooth low energy. Thus the combined-device node (i.e., “intermediate communication device+voice unit”) can be considered a node through the linked operation of both devices and their interaction with other group members and a group messaging service. Implementations that do not use a voice unit or similar end user device are available, but in some of those implementations certain advantages (e.g., a simple talk/listen unit) may be unavailable.

In essence, a communication node in various implementations is a communication endpoint. That endpoint can be used by a human user, a bot, a server implementing a group messaging service or another group messaging participant. FIG. 2 illustrates several implementations of a communication node 201. In non-limiting Example 1, node 201A comprises an intermediate communication device 205 linked to an end user device 214 (e.g., a voice unit), though voice unit 234 might still be linked or otherwise connected to another device via Bluetooth or the like. Non-limiting Example 2 of FIG. 2 illustrates a voice unit 234 alone as a communication node 201B. Non-limiting Example 3 of FIG. 2 shows a node 201C comprising an intermediate communication device 225 alone (though it might still be linked or otherwise connected to another device via Bluetooth or the like). Non-limiting Example 4 of FIG. 2 illustrates a basic communication node 201D, which comprises some type of hardware device 245 (e.g., a server, a communication device, a computing system) on which software 208 is running to execute the communication application that permits group messaging. Non-limiting Example 5 of FIG. 2 illustrates a bot node 201E, which comprises some type of application 288 or other function performed by the bot.

User node 201 may represent one of the communication nodes 104, 106, 108 of FIG. 1, server system 141 of FIG. 1, one of the communication devices 112, 122, 132 of FIG. 1, one of the end user devices 110, 120, 130 of ff1, or another communication node implementation such as any user nodes, bot nodes and/or others discussed herein).

Non-limiting Example 1 of FIG. 2 comprises a user node 201A comprising a distinct end user device 214 (e.g., a voice unit) that permits voice communication with other communication group members via an intermediate communication device 205 (e.g., a wireless communication device). In some implementations, end user device 214 can be a personal (e.g., wearable), push-to-talk (PTT) electronic device that transmits messages through an intermediate communication device such as a smartphone, cellphone, tablet, computer, gaming device, laptop computer, or some other communication device capable of communicating using packet networks or some other communication network. In some implementations a voice unit 214 or the like is connected only by Bluetooth (e.g., Bluetooth LE) to the ICD 205, on which an application is executed that addresses all of the other communication and data management functions.

In the illustrated Example 1, node 201A further comprises processing system 202 and communication interface system 210. Processing system 202 further comprises processing circuitry 204 and storage system 206. Processing circuitry 204 can comprise one or more processors, microprocessors and/or other circuitry that retrieves and executes software 208 from storage system 206. Processing circuitry 204 may be embedded in various types of equipment.

Storage system 206 comprises a non-transitory computer readable storage medium, such as a disk drive, flash drive, data storage circuitry, or some other hardware memory apparatus. Storage system 206 also may be embedded in various types of equipment. In some examples, a computer apparatus and/or computing system could comprise processing circuitry 204, storage system 206 and software 208. Software 208 in some implementations can comprise one or more modules, which may be part of or supplemental to communication software that enables group members' communication with one another. The modules may be configured to operate in a manner that carries out one or more of the processes, methods, etc. described in connection with one or more implementations described herein. In addition, software 208 may include operating systems, utilities, drivers, network interfaces, applications, or some other type of software.

Communication interface system 210 further comprises transceiver 212 for communicating with device 214. Transceiver 212 can comprise communication components, such as ports, signal processing circuitry, memory, software, and the like. Transceiver 212 communicates with device 214 over a link that may comprise a Bluetooth communication link, Bluetooth low energy, WiFi link, infrared, ultrasonic or any other suitable communication link between intermediate communication device 205 and device 214. In some implementations all network communications are Internet protocol (IP) communications in that they travel over the Internet from end to end.

Non-limiting Example 2 illustrates an end user device 234 that can operate as a voice unit or the like in some implementations and operates as a stand-alone device without the need for an intermediate communication device to transmit message to and receive messages from a communication network. Again, this end user device 234 may be a portable, wearable, PTT device that permits simple voice communication between users of a given communication network. Device 234 can connect to a group messaging service using a Long-Term Evolution (LTE) chip or the like (thus being able to reach the Internet without the need for an ICD), but which does not require the intermediary operation of another device. This type of device similarly can be equipped with a WiFi chip that allows it to operate if is within range of an appropriate WiFi connection point. Such a stand-alone end user device also can include an ARM-based CPU that can run a full application and stack, albeit on a less robust device and/or system than some other node examples. Unlike a voice unit that is paired to run with and requires the intermediate use of an ICD, device 234 can run the application that would otherwise be run on the intermediate communication device in a paired-device node configuration.

Non-limiting Example 3 illustrates a personal communication node 201C that also does not employ multiple distinct devices, but instead utilizes a single communication device (which may be wireless in some implementations), such as a personal communication user node that contains and includes all necessary wireless communication interfaces and processing resources, among other features. The communication device 225 can be a smartphone, cellphone, etc. that has a more robust operational and hardware functionality than a device like that described in connection with node Example 2. Furthermore, software 208 usable in these and other examples can comprise a virtual machine that is executed on a computing device, including virtual devices or software executed by a virtualized processing system or virtualized computing system. Non-limiting Example 3 of FIG. 2 illustrates personal communication node software within an operating environment of an electronic device, wherein the electronic device may comprise a smartphone, cellphone, tablet device, computer, gaming device, laptop computer, or some other communication device capable of communicating using packet networks or some other communication network, running a personal communication node software application that comprises personal communication node 201.

In the illustrated Example 3, node 201C may comprise communication device 225, which comprises a processing system further comprising processing circuitry and a storage system and which further comprises components, features and operational characteristics similar to those of intermediate communication device 205.

Non-limiting Example 4 of FIG. 2 illustrates a device 245 on which software 208 is running to execute the communication application that permits group messaging. Device 245 can be a server, a communication device, a computing system or other device or combination of devices that is able to perform the functions described herein.

Non-limiting Example 5 of FIG. 2 is a bot node 209 configured to perform an application (e.g., application 288 of FIG. 2) and to be linked to group members and/or other components of a communication system as described herein.

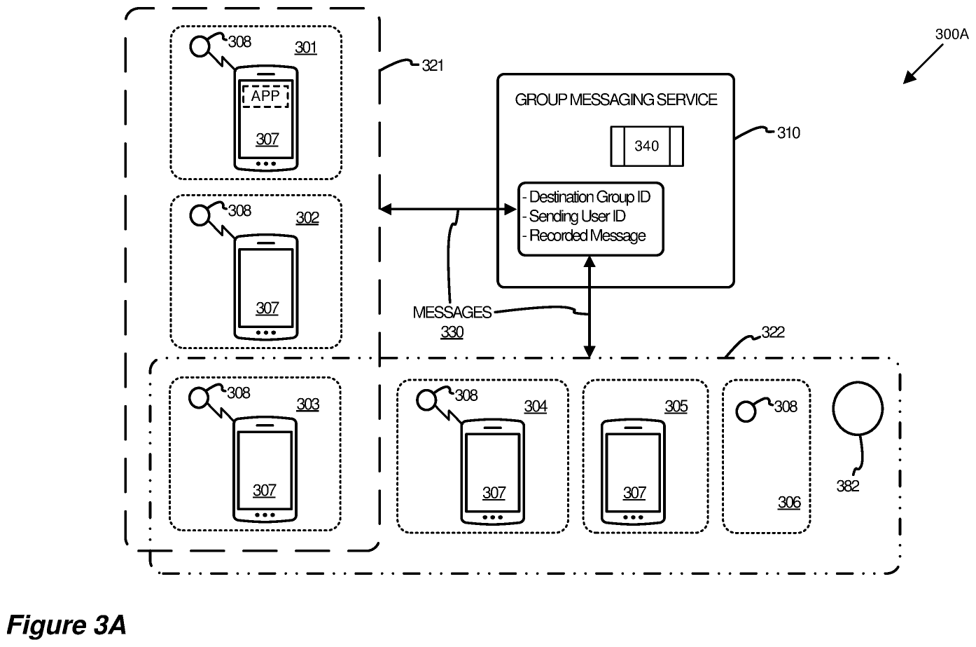

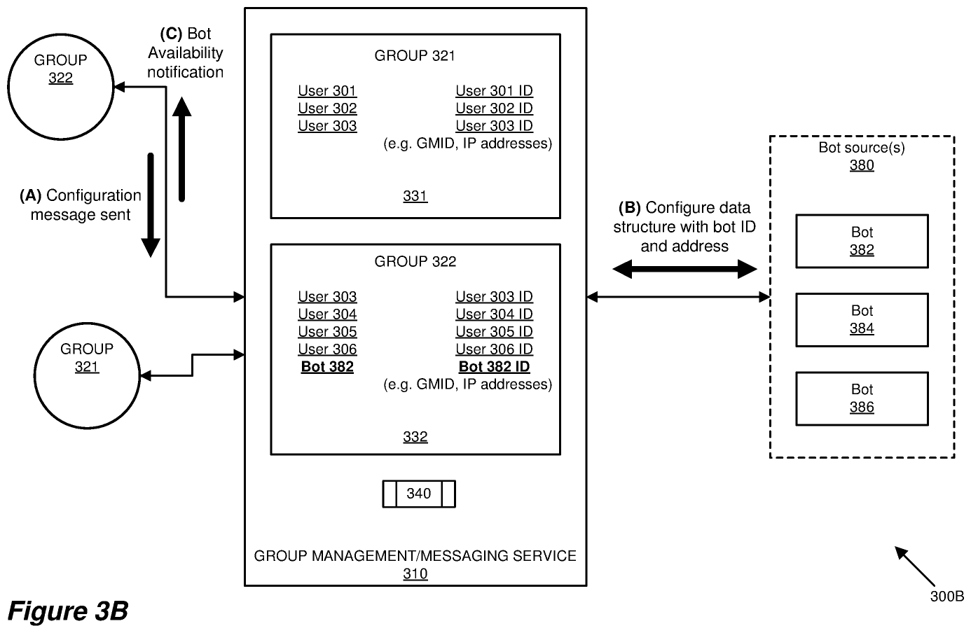

FIG. 3A illustrates a basic group messaging system 300A (also referred to as a “group management service” in some implementations) that can be utilized in connection with implementations of emergency support utilizing group communications including use of one or more support bots in connection with a group communication service. Group messaging system 300A includes at least communication nodes 301, 302, 303, 304, 305, 306, a bot node 382, and a group messaging service 310. Group messaging service 310 includes one or more computers and/or servers that manage and facilitate communications among and between the communication nodes. Service 310 can be operated as a communication service that operates on the IP layer above the transport (CDMA, LTE, etc.).

Each communication node 301-304 includes a voice unit 308 and an intermediate communication device 307 paired to the voice unit 308 and running a communication application that enable communication with group messaging service 310 and members of any group(s) to which a user is assigned. Each intermediate communication device can be a smartphone, cellphone, tablet device, computer, gaming device, laptop computer, or some other communication device capable of communicating using packet networks or some other communication network, running a personal communication node software application. In some embodiments, user nodes 301-304 include a wearable pendant 308 for wireless bidirectional audio (e.g., voice) communication with a linked mobile device 307. In some implementations, the wireless bidirectional communication protocol linking a device 307 to pendant 308 uses Bluetooth LE. Mobile devices 307 may also include one or more embedded or downloadable software applications that facilitate audio communication between the wearable pendant 116 and other user nodes 301-305 and/or group messaging service 310.

In addition to two-device nodes 301-304, group 322 also includes two other user nodes 305, 306 and a bot node 319. In the absence of a wearable pendant or other end user device, the user for node 305 may communicate with the mobile device 307 as they would with any smartphone or similar mobile device. User node 306 is a stand-alone end user device 308 that does not use an intermediate communication device to communicate with other nodes and the group messaging service 310. In some implementations, node 306 can include a device that may or may not include a screen and that can connect to the group messaging service 310 using an LTE chip or the like, thus not requiring intermediary operation of another device. Such a stand-alone end user device also can include an ARM-based CPU that can run a full application and stack. Finally, group 322 further includes an optional bot node 382, which can be added, removed, modified and enabled to operate in one or more modes and with one or more capabilities as described herein.

A given user node may be in none, one, or any number of groups serviced by group messaging service 310. Moreover, multiple groups may include different user nodes, or exactly the same user nodes. There is no limit to the association between user nodes and groups. User nodes 301-305 may be organized into one or more groups 321, 322, where each group includes at least one user node (in the non-limiting example of groups 321, 322, node 303 is a member of both groups). In the non-limiting exemplary implementation illustrated in FIG. 3A, groups 321, 322 are serviced by group messaging system 310. Group 321 includes three user nodes 301, 302, 303, each of which includes a wearable pendant 308. When a user node of a group communicates with other members of that group through service 310, messages 330 can be sent wirelessly and through an appropriate network (e.g., the Internet) between the communicating user nodes and service 310. Messages 330 may include the following: all or part of a recorded audio message; a sending user nodes identification (ID); and a group ID for the destination group to which the message is being delivered. Service 310 receives a message 330, identifies the group ID associated with the message 330, looks up the user nodes contained within the destination group, and transmits the message 330 including the ID of the sending user node to each user node in the destination group. For example, if user of user node 302 wants to send a message 330 to the other members of group 321, the user of node 302 speaks into her wearable pendant 308 and identifies group 321 as the destination for the message 330 while speaking to generate the message into the wearable pendant 308. In some cases, the destination group may be predetermined and thus identification by a user unnecessary for every voice communication generated.

An application on mobile device 307 of node 302 then creates a message 330 including the audio data created by the user and sends the message 330 to the group messaging service 310. The software application on device 307 of node 302 may have been previously configured with group messaging service 310, which receives, analyzes, and transmits the message 330 to each of the nodes in the destination group 321. Each receiving user node 301, 303 of group 321 receives and hears the audio for the message 330 sent by node 302. Use of groups as described herein does not preclude sending messages directly from one user node to a different user node. Moreover, in some implementations, messages may be sent to and/or received from a group to which the receiver and/or sender do not belong.

FIG. 3B illustrates one or more messaging flows for configuring bot 382 into group 322 of FIG. 3A in accordance with one or more implementations of emergency support utilizing group communications herein. System 300B includes groups 321, 322 as described with reference to FIG. 3A, group messaging service 310, and one or more existing bots 382, 384, 386 available from one or more bot sources 380. Bots 382, 384, 386 are software applications for performing one or more tasks over the Internet. Therefore, bots 382, 384, 386 are Internet-connected (or otherwise network-connected) and in some implementations are separate from the group messaging service 310. System 300B (which may be the same system 300A from FIG. 3A in some implementations) operates in a manner similar to an instant messaging application, service, etc.

The communication application used by user nodes may connect to the group messaging service 310 on the Internet and performs variation functions in connection with service 310 (e.g., verifying a particular user node's identity (also sometimes referred to as authentication) and sending messages between users (sometimes based on communication group affiliation or membership)). When a user logs in or otherwise initiates communication with group messaging service 310, the user can provide an identifier and a password or other verification. The identifier can be identifying data (e.g., an IP address, port, group messaging service identification (GMID), and/or other data) that allows the group messaging service 310 to identify and track activity for the identifying user or other node. When a user (or bot) is identified as being a member of a given group, that member's identifying data can be placed in a specified data structure or the like. As users log on and off of system 300B, they may be added to and removed from the groups to which they belong. Also, users and bots may be added or removed based on the creation, termination and changes to one or more communication groups.

Bots 208, like users, may be incorporated into communication groups by configuring an available bot 382, 384, 386 in the group messaging service 310 in a similar fashion to user nodes 301-306. Service 310 in system 300B can utilize data structures 331, 332 for specifying which user nodes 301-306 and bots 382, 384, 386 are in each group 321, 322, respectively. Each such data structure 331, 332 includes identifiers and addresses for each user node 301-306 and any member bot (e.g., one of bot 382, 384, 386 from source 380) in the relevant data structure. Data structure 331 includes identifiers and addresses for each of the user nodes 301-303 in group 321, while data structure 332 includes identifiers and addresses for each user node 303-306 and bot 382 (after it is added) in group 322. Group messaging service 310 may include any number of data structures, and a given bot 382, 384, 386 may be a member of any number of data structures. Data structures may include service IDs or identifiers for any user nodes as well. Service IDs may be anything, including a user name, group messaging ID, and/or email address, or anything else that assists in uniquely identifying a user or user node.

Group messaging service 310 or a user of service 310 can initiate adding a bot 382 to a group 322. In some cases, a bot may be added to one or more communication groups based on a triggering event, such as the creation of a specialized or special-purpose bot (e.g., in connection with a hazardous material response, a patient care triage response, etc.). In the non-limiting example of FIG. 3B, one operational sequence of which is designated by the reference letters (A) through (C), though these and/or other steps could be performed in any order in various implementations, a user in group 322 sends a configuration message (A) to service 310 through the application on a corresponding mobile device, requesting a specific bot 382 the user has identified and is requesting to be added to group 322. The user might not have the address of the requested bot 382, but is at least able to specify the requested bot. It is assumed that multiple existing bots may be available (e.g., from one or more sources 380) to be messaged, such as bots 382, 384, 386 of FIG. 3B.

In response to service 310 receiving message (A) for bot 382, data structure 332 is configured (B) to route messages to or from bot 382. Configuration can be performed automatically by software (e.g., software 340) or can be performed by a system administrator or the like. An address and identifier for bot 382 is added to data structure 332 for group 322. In some embodiments, the system administrator may need to obtain information associated with bot 382 in order to configure data structure 332. Once configuration of bot 382 is completed, members of group 322 can be sent a message or provided other notification (C) of the bot's membership and availability. Rather than (or in addition to) a message, the bot may appear on a user interface of the communication group's relevant devices. At this point, bot 382 is configured into group 322 along with user nodes 303-306 and is ready to receive messages from and send messages to any user node in group 322. Group messaging service 310 also includes a process (e.g., implemented by software 340) for sending messages to a bot configured in a group. Applications, software, processes, methods, etc. described herein may be stored and/or executed in a single device or in a combination of devices, including in distributed computing arrangements involving multiple devices, systems, etc.

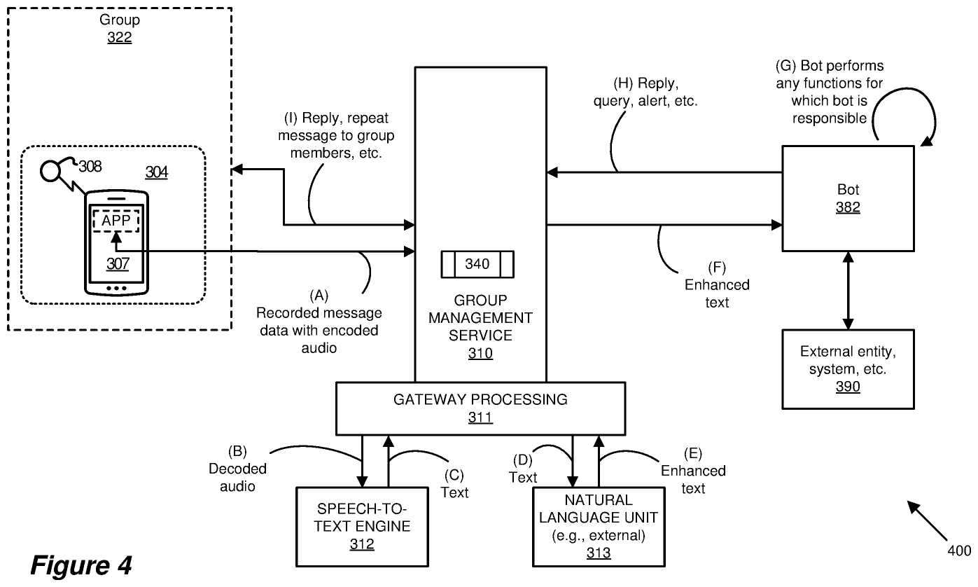

FIG. 4 illustrates a system 400 that can be utilized for performing one or more implementations of emergency support utilizing group communications that include bot participation, one operational sequence of which is designated by the reference letters (A) through (I), though these and/or other steps could be performed in any order in various implementations. Functional block diagrams, operational scenarios and sequences, and flow diagrams provided in the Figures are representative of exemplary systems, environments, and methodologies for performing aspects of the disclosure. While, for purposes of simplicity of explanation, methods included herein may be in the form of a functional diagram, operational scenario or sequence, or flow diagram, and may be described as a series of acts, it is to be understood and appreciated that the methods are not limited by the order of acts, as some acts may, in accordance therewith, occur in a different order and/or concurrently with other acts from that shown and described herein. For example, those skilled in the art will understand and appreciate that a method could alternatively be represented as a series of interrelated states or events, such as in a state diagram. Moreover, not all acts illustrated in a methodology may be required for a novel implementation.

Once a group messaging service 310 has been configured to use one or more bots 382, user nodes (e.g., node 304) within group 322 may send and receive messages from the bots 382 and provide information and data that can be used by the bot 382 in connection with communications with other entities, systems, etc. (e.g., external entity 390). In some implementations bot 382 may be configured to respond to enhanced text rather than recorded messages generated by user nodes in group 322 (e.g., using voice units and the like). In such cases, group messaging service 310 is able to convert audio from users' recorded messages into enhanced text which is sent to bot 382 to execute one or more actions.

Group 322 includes user node 304, which itself comprises mobile device 307 and wearable pendant 308 (a user node in this type of setting may also be an intermediate communication device executing a similar application and/or a stand-alone end user device executing a similar application). User node 304 sends a recorded message (A) with encoded audio to the group messaging service 310, where the recorded message is directed to a bot 382 that is a member of group 322. Group messaging service 310 decodes the encoded audio in the recorded message to create decoded audio. Group messaging service 310 can execute process 340 to identify bot 382, a voice library and any other resources required for execution, and then directs the recorded message to users and bots appropriately, for example, repeating the recorded message to members of group 322.

Group messaging service 310 either includes a voice library or has access to one or more remote voice libraries. Voice libraries include at least a speech-to-text engine 312 and a natural language unit 313, where the speech-to-text engine is generally configured to convert voice message audio data to text data that can be further processed and/or evaluated for content. In some implementations the speech-to-text engine 312 is part of group messaging service 310 while natural language unit 313 is external. In other implementations the speech-to-text engine 312 is remote while the natural language unit 313 is part of service 310. Remote portions of the voice library can be accessed through gateway processing 311. Data structures of the group messaging service 310 need to be configured as to whether or not voice libraries are required to be used for group messages. For example, some bots may require a voice library to be used while other bots may not.

Service 310 sends decoded audio (B) to the speech-to-text engine 312, which converts the decoded audio into text (C) that is sent to the group messaging service 310. In some embodiments, the speech-to-text engine 312 sends the text (D) directly to the natural language unit 313. After receiving the text, group messaging service 310 sends the text to the natural language unit 313, which reviews the text and converts the text into enhanced text. Enhanced text is clarified and simplified from text into a form more suitable for presentation to bot 382 to execute. The natural language unit 313 sends the enhanced text (E) to service 310.

After receiving the enhanced text from the natural language unit 313, the group messaging service 310 sends the enhanced text (F) to the selected bot 382 corresponding to the recorded message. Bot 382 receives the enhanced text and in response, performs one or more designated actions (G) based on the decoded audio and enhanced text. In some implementations bot 382 is associated with one or more external entities, systems, services, etc. (e.g., one of those described herein in connection with hazardous materials responses, triaging, etc.).

In response to receiving a reply, query, alert (H) or other communication from the bot 382, group messaging service 310 forwards an appropriate communication (I) to group 322 or selected members of the group. In some implementations service 310 provides the original recorded message to the group 322 in order to inform other user nodes of the message sent by user node 304.

In some implementations, each end user device (including a stand-alone end user device) can be implemented in a “push-to-talk” operational mode that allows an end user to press a transmit toggle button or the like (e.g., by pushing and holding a toggle) to initiate sending a voice communication to one or more nodes in the communication group. While the toggle is in its “transmit” position, an end user device is configured to collect and transmit audio data from the user (e.g., recording voice communications). This can be done in a variety of ways. The collected audio data can be processed and subsequently transmitted by the end user device or by a linked intermediate communication device (e.g., a smartphone, cellphone, gaming device, tablet, or laptop). When the toggle is switched back to its “receive” position, recording stops. Any collected audio data is transmitted either continuously, in segments or as a whole to the one or more communication group members (and any other destination(s) for the audio data). The collected audio data can be transmitted using any appropriate transmission scheme. In one non-limiting example discussed below, audio data collected by an end user device can be transmitted to its linked intermediate communication device via an appropriate Bluetooth mode. Likewise, audio data collected by an intermediate communication device can be sent over a broader network using any appropriate communication protocol or scheme.

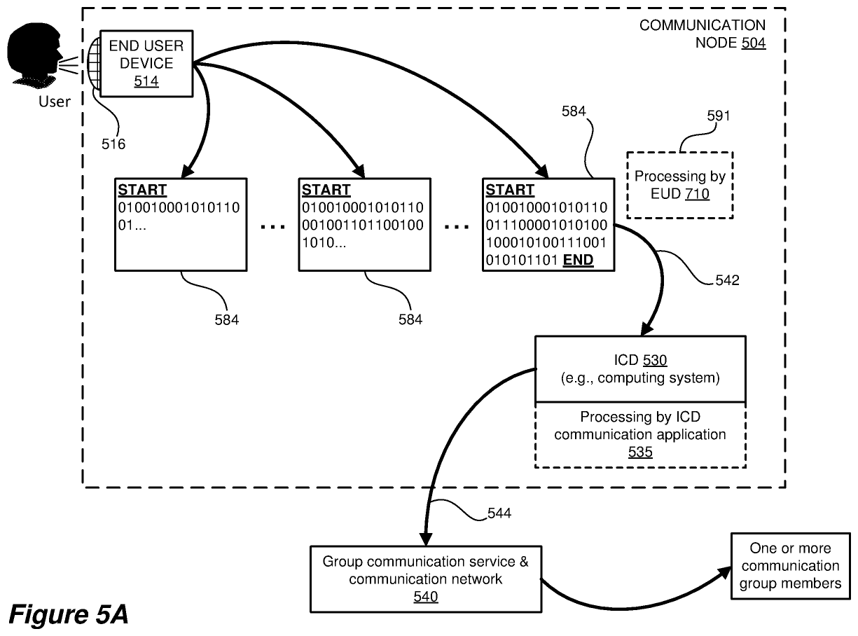

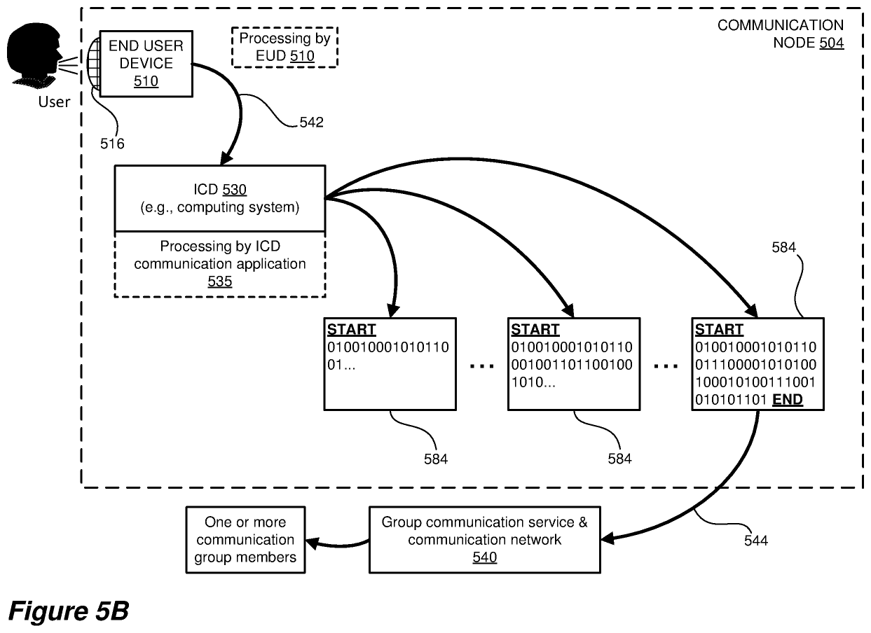

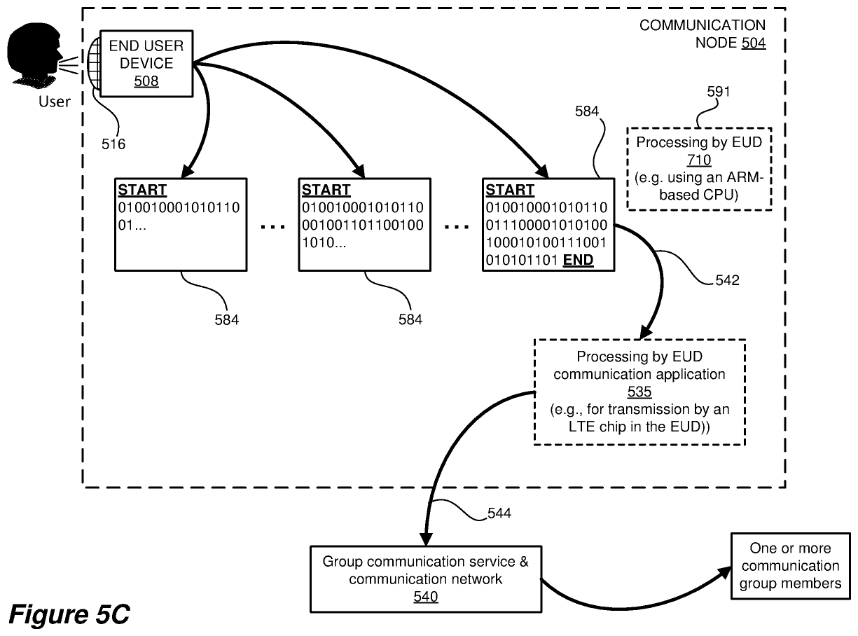

In one implementation, a non-limiting example of which is illustrated in FIG. 5A, a communication node 504 includes an end user device 510 (e.g., a voice unit of FIG. 1, and/or a device 308 of FIG. 3A) that has a microphone 516 configured to collect audio data from a human user. As illustrated in FIG. 5A, the end user device 510 begins storing the collected audio data in a memory location 584. This audio data collection process continues until a push-to-talk button on end user device 510 is released (i.e., the END signal in FIG. 5A). Some additional processing 591 may be performed by end user device 510 before the collected audio data is transmitted at 542 to an intermediate communication device 530 that also is part of communication node 504. Again, some additional processing 535 may be performed by ICD 530 before it transmits at 544 the audio data to one or more additional communication group members via group communication service and communication network 540. In some implementations, multiple members of a group can be collecting and transmitting audio data.

In another non-limiting example shown in FIG. 5B, it is the intermediate communication device 530 that stores all or segments of the collected audio data before it is transmitted via service and network 540. The end user device 510 may process audio data collected from a user prior to transmission at 543 to the ICD 530 (e.g., the collected audio data may be encrypted, buffered to permit error correction, assembled into packets, etc.). The intermediate communication device 530 builds the audio data until the push-to-talk button on the EUD 510 is switched back to receive, at which point the ICD 530 can transmit the collected audio data to group communication service and network 540 and thus to one or more communication group members or the like.

In another non-limiting example shown in FIG. 5C, the end user device 514 is a stand-alone communication device (e.g., a device 308 in node 306 of FIG. 3A) that has onboard capabilities to collect, process and transmit audio data from a human user. In some implementations device 516 can use an ARM-based CPU and/or related components to provide basic capabilities as described herein. As illustrated in FIG. 5C, end user device 516 begins storing the collected audio data in a memory location 584. Again, this audio data collection process can continue until a push-to-talk button on end user device 508 is released (i.e., the END signal in FIG. 5C). Additional processing 591 may be performed by end user device 508 before the collected audio data is transmitted at 542 to communication application that also is executed by end user device 508. Again, some additional processing 535 may be performed by device 508 before it transmits at 544 the audio data to one or more additional communication group members via group communication service and communication network 540.

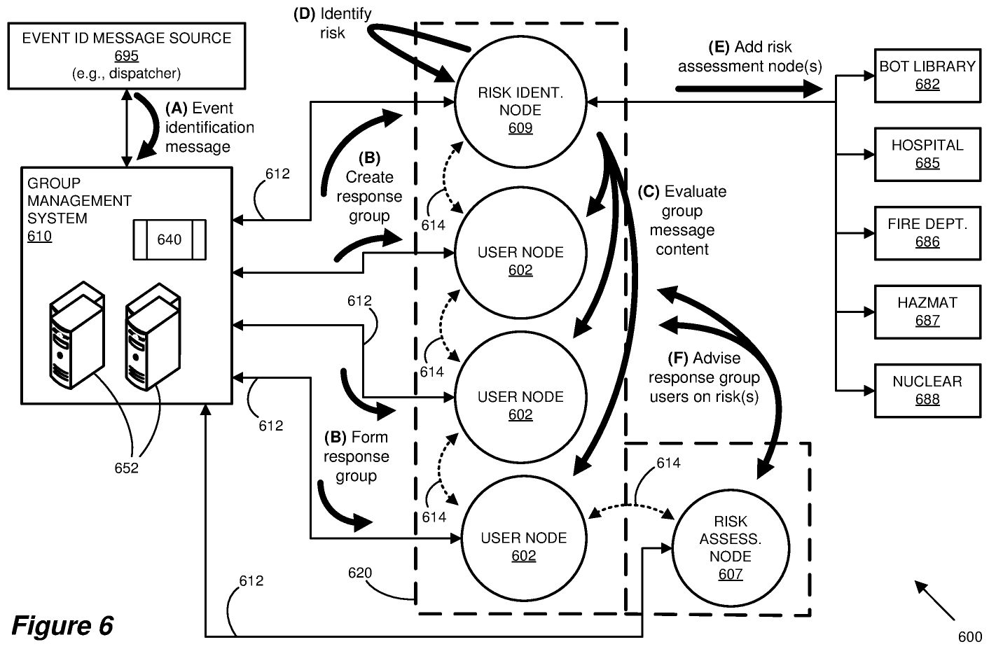

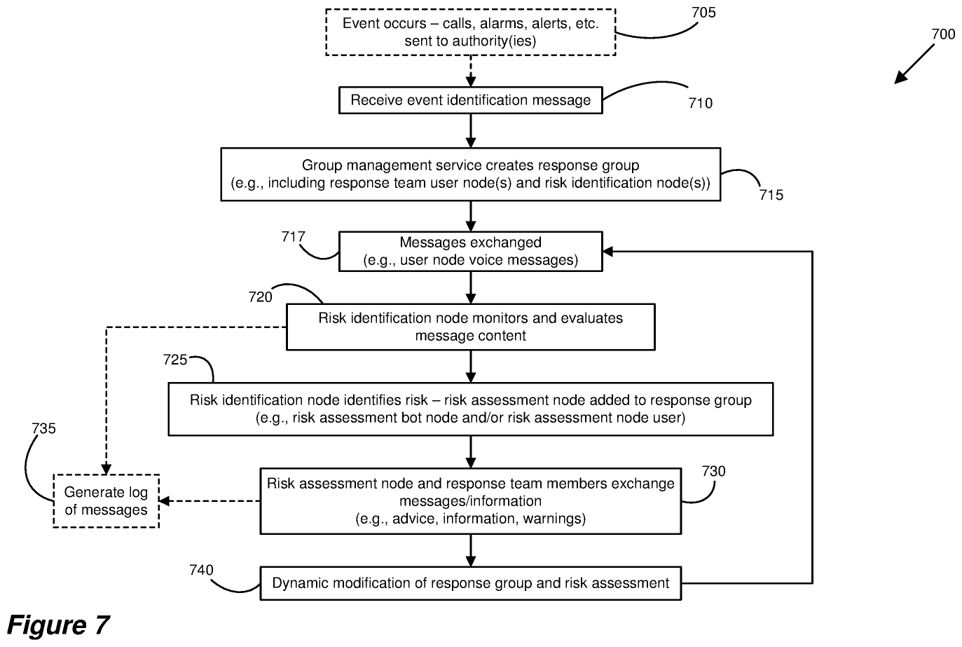

Non-limiting exemplary bot support in emergency services group communications is shown through operation of system 600 is illustrated in FIG. 6, one operational sequence of which is designated by the reference letters (A) through (F), but note that these and/or other steps could be performed in any order in various implementations. Group management system 610 (e.g., a computing system comprising one or more servers 652 or the like and running software or an application 640, which may in some implementations be the same or similar to process 700 of FIG. 7) receives (A) an event identification message from an event identification message source, identifying an event requiring emergency response assistance. The message source 695 could be an alarm, a 9-1-1 call, a police alert, a police or fire department call or some other communication originating from a public safety authority or the like. If the group management system 610 requires clarification or further information regarding the incident, system 610 can request such information from source 695.

A response node group 620 is then created (B) by the group management system 610. Response node group 620 includes one or more response team user nodes 602 and one or more risk identification nodes 609, and the member nodes of group 620 may communicate with one another via other appropriate links 612, 614 either via group management system 610 or directly with one another. Response team user nodes 602 are configured to allow users to send messages between and among the members of group 620 during the response of group 620 to the signaled emergency. These messages are monitored in FIG. 6 by risk identification node 609, which may be a bot node or a user node, or a combination of both. Risk identification node 609 monitors and evaluates (C) the content of the group's messages to determine whether specific types of risks (e.g., actual or potential threats to safety, hazards, etc.) are present or arise that were not previously anticipated. For example, in responding to a fire call, the firefighters may find that they are also facing a hazardous materials issue or they may find that there are injured people previously unknown to the response network. In such cases, the content of the messages sent by user nodes 602 of group 620 will be screened and evaluated, for example by a risk identification node module that is configured to detect terminology and conditions that evince the presence of a specific type of risk. In some implementations a gateway processing system, such as the non-limiting example of system 311 of FIG. 4, can be utilized to facilitate bot node evaluation of message content and redirection of message content and/or other communications to the appropriate external entity, system, etc. that can assist group 620 and further assist group management system 610 and/or a risk identification node 609 in the dynamic formation and membership modifications of one or more response node groups.

Once a specific type of risk is identified (D), risk identification node 609 can then access resources to assist the response node group. In the non-limiting example of FIG. 6, risk identification node 609 can access bots and/or personnel that are affiliated with a hospital 685, a fire department 686, a hazardous materials (“hazmat”) agency 687 and/or a radioactive/nuclear materials agency 688. Also, a risk identification node 609 may be able to access a bot library 682 or other source of bots and, based on an identified risk, select one or more risk assessment bot node that can assist response node group team users in evaluating and neutralizing a newly-detected risk. A risk-specific risk assessment node bot and/or user node can then be added (E) to response node group 620 by the risk identification node 609 (or by group management system 610 or another actor/component of system 600). The response node group 620 now includes new risk assessment node 607, which can advise (F) response node group users via node 602, for example providing advice, warnings, information, etc. either using audio or through a user interface, if available. Moreover, risk assessment bot node 607 can be configured to pose questions to prompt a user to provide additional information to assist in identifying, mitigating, evaluating, etc. a risk. A pre-scripted checklist and sequencing of hazmat and other data collection questions can provide a better organized and more efficient data collection and risk assessment process for the entire response node group without requiring the response node group users to have to remember or refer to information about potential risks in the midst of their response work. Artificial intelligence (AI) can be employed (e.g., as part of software or one or more processes or applications (e.g. 640 of FIG. 6, or 700 of FIG. 7), which can be executed on one or more computing systems 652 in system 600 of FIG. 6) to obtain voice-driven data and to determine whether specific steps and/or processes should be executed (e.g., precautions to be taken), as well as monitoring developing risk identifications and assessments.

Referring to FIG. 7, a method 700 of providing bot support in emergency services group communications to a response team in an emergency response in a distributed group communication environment is shown. Method 700 (which may be the same or similar to software or application 640 of FIG. 6 in some implementations) is described with reference to exemplary elements, apparatus, systems and other information from FIG. 6 and other Figures in some implementations. The description below references operations of FIG. 7 parenthetically.

As described in connection with FIG. 6, an event occurs and a call for assistance is made in response to an event (705), for example through calls, alarms, alerts and other communications sent to and from authorities and the like. An event identification message is then sent (710). A group management service creates (715) a response node group that responds to the event notification, where the group is created so that the team members can exchange messages (717) in the course of responding to the identified event (e.g., exchanging voice messages, though text and other types of messages can be exchanged and evaluated by the risk identification bot node(s)). In some implementations the response node group includes one or more response team members who each can operate a response team user node. Additionally, one or more risk identification nodes are included in the response node group. In some implementations each risk identification node is a bot node that receives all of the voice and other messages exchanged by response team members so that the messages' content can be evaluated (720). This monitoring and evaluation function by the risk identification node allows it to detect a risk that the response node group may not have known about previously. The risk identification node can also be a user node or a node that combines a user and a bot in performing the monitoring and evaluation functions.

While monitoring and evaluating the voice messages exchanged by response node group user nodes, a risk is identified (725) and a risk assessment node is added to the response node group (e.g., via dynamic node group modification by a group management service or the like and/or by the risk identification node). The risk assessment node that is added can be a bot node or a user node, or a combination of both, that has information (and perhaps experience) with the specific type of risk identified by the risk identification node. The risk assessment node can then exchange (730) messages, information, advice, warnings and any other information that might helpful to the response node group users (i.e., the response team). In some implementations those communications from the risk assessment node may be audio/voice transmissions, though it is possible in some implementations to also have graphic and/or other types of information transmitted to the response team. The type of information transmitted may depend on the type of communication equipment being used by the response team and the team members' ability to communicate in a manner other than by voice.

As messages and information are transmitted among and between response node group nodes, including the risk identification node and any risk assessment node(s) added to the response node group, a log of messages and other information may be generated (735). This might be a requirement with regard to the response to certain types of emergencies, or it might be elective for an agency or other organization to maintain such logs for training and other purposes. As messages and/or information are exchanged, process 700 can dynamically modify (740) the response node group and/or risk assessment (e.g., adding user and/or bot nodes, removing user and/or bot nodes, connecting one or more other groups, etc.).

In an emergency setting, multiple victims may require evaluation prior to being transported for medical attention beyond what is available at the site of the emergency. Triage is the prioritization of patient care and treatment based on the nature, number and severity of the patients' injuries and conditions. Where multiple responders participate in patient triage, the relative prioritization of patients across the entire incident may be difficult due to distance, geography, responding agency differences, environmental circumstances (e.g., the presence of debris, fire, smoke, etc.), noise and even language differences between responders. In some implementations of bot support in triage group communications, one or more triage groups are utilized to enable communications among and between responders to an emergency who are performing triage for patients while also facilitating triage of patients that can be faster, more thorough and prioritized across multiple responders and, in some implementations, multiple response teams.

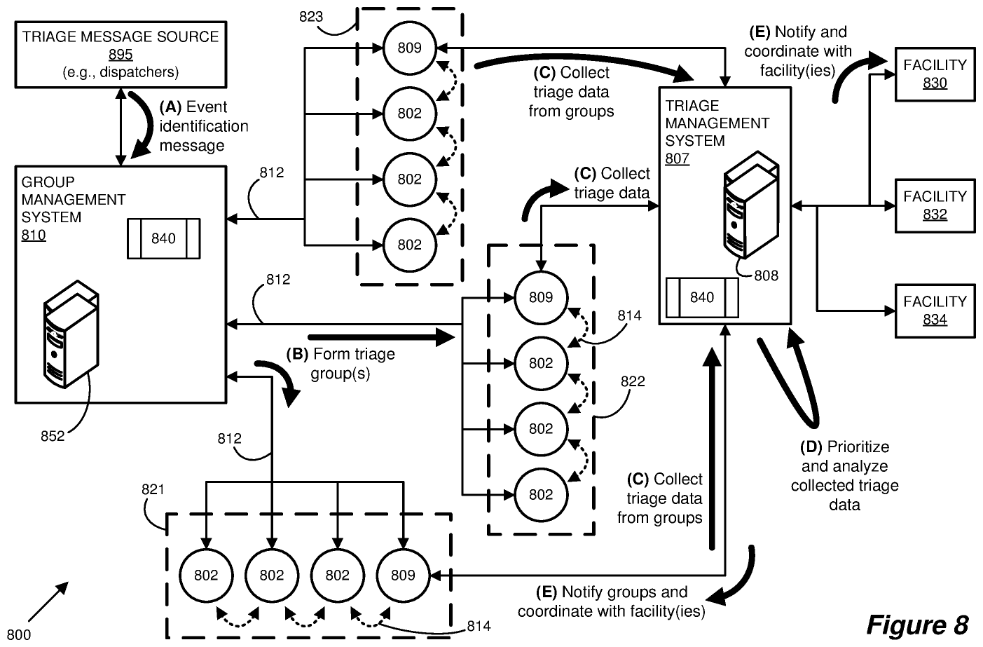

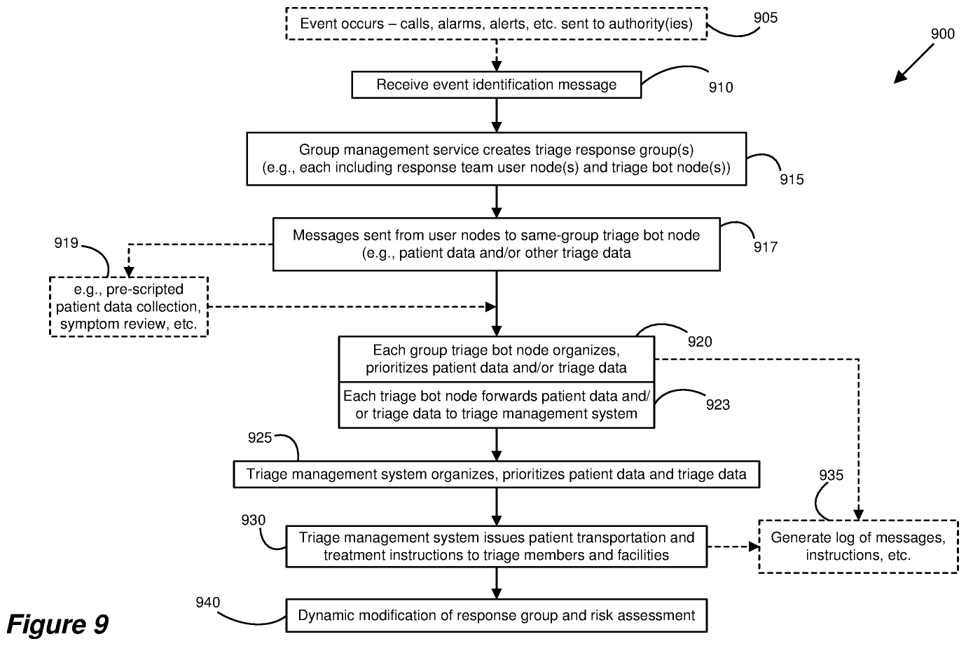

The one or more exemplary systems 800 illustrated in FIG. 8 show non-limiting exemplary implementations of bot support in triage group communications, one operational sequence of which is designated by the reference letters (A) through (E), though these and/or other steps could be performed in any order in various implementations. As seen in FIG. 8, a group management system 810 (e.g., a computing system comprising one or more servers 852 or the like or the like and running software or an application 840, which may in some implementations be the same or similar to process 900 of FIG. 9), may receive (A) one or more event identification messages or responder call messages from a message source 895, identifying an event requiring emergency response assistance involving victims who may require medical attention. The message source 895 could be an alarm, a 9-1-1 call, a police alert, a police or fire department call or some other communication originating from a public safety authority or the like. If the group management system 810 requires clarification or further information regarding the incident, system 810 can request such information from source 895.

In the non-limiting example of FIG. 8, multiple triage node groups 821, 822, 823 are then created (B) by the group management system 810. Triage node groups 821-823 can each include one or more response team user nodes 802 and a triage bot node 809. The nodes 802, 809 of each group 821-823 may communicate with one another via other appropriate links 812, 814 either via group management system 810 or directly with one another. Response team user nodes 802 are configured to allow users to send messages (e.g., voice or other audio) between and among the members of each communication group 821-823 during the multi-group response to the signaled emergency. These messages are monitored by triage bot nodes 809. In some implementations the triage bot nodes 809 can collect these messages as patient data and/or other triage data that can be processed by either the triage bot node 809 and/or a triage management system 807 or the like.

In some implementations the non-limiting exemplary system(s) of FIG. 8 can be scaled to include more than three communication groups managed by group management system 810. Also, there may be multiple groups managed by multiple group management systems similar to system 810, where these group management systems coordinate their triage data flows to appropriate locations (e.g., hospitals). Moreover, the system 800 of FIG. 8 may be “reverse scaled” so that only one communication group is created by group management system 810, but the single created group has many user nodes 802 that can communicate with one or more triage bot nodes (that might also have membership in the single communication group). Other topographies and organizational schemes can be implemented as well. Group management system 810 can dynamically modify the triage groups 821, 822, 823 with regard to membership, operational characteristics, routing of patient and/or other triage data, and in other ways to achieve desired performance characteristics of system 800.

Each triage bot node 809 can monitor and evaluate triage data sent by the various user nodes 802. In some implementations each triage bot node 809 receives voice communications from user nodes 802 regarding the status of and/or data about a given victim or other subject. Thus “virtual triage stations” can be implemented that do not require physically moving each human subject to a given physical location nor having central physical triage locations as has been done in some traditional triage arrangements. A triage bot node 809 may utilize a triage approach that is suitable to the type of emergency, the type of responder participating in the triage effort, and other factors. Non-limiting examples of triage approaches implementable include the START (simple triage and rapid treatment) system, color-coded systems, and even more simple classifications (e.g., likely to live regardless of care; unlikely to live regardless of care; and possible difference based on provision of immediate care).

In many current emergency medical services (EMS) systems, such a simple model may still be applied. In early stages of an emergency, practical limitations sometimes demand that a simple approach be used. However, using implementations of bot support in triage group communications, more “effective” responders may be available due to the virtual nature of one or more triage stations. Such implementations therefore can, in some settings, permit a more granular or specific triage system to be utilized despite a relatively small number of responders, a relatively large number of patients, a widely dispersed area to which a response is needed, etc.

The triage bot nodes can be connected to and collect triage data to be sent (C) to a triage management system 807 (e.g., a computing system comprising one or more servers 808 or the like and running software or an application 840 in some implementations, which may in some implementations be the same or similar to process 900 of FIG. 9), as seen in FIG. 8. Triage management system 807 may be contained within group management system 810 in some implementations, or may be a separate operation managed by the same or a different authority than the group management system 810. Group management system 810 and triage management system 807 can perform one or more processes, methods, etc. disclosed herein operating in concert (e.g., software/application 840 in some implementations) as either a single system (collocated or distributed) or as two coordinated systems. Triage management system 807 can in turn be connected to multiple facilities 830 that are capable of responding to different types of urgent and/or other medical care and treatment. For example, triage management system 807 of FIG. 8 may be connected to one or more local hospitals, a burn center, a surgical specialty center, a helicopter or other special transport system, etc.

In some implementations the triage bot nodes 809 can be “passive,” acting to receive and sort triage data from user nodes 802. The triage bot nodes 809 can then prioritize communications and/or other responsive actions based on the triage data received (for example, ensuring that critical data regarding patients requiring more immediate evacuation and/or medical treatment is sent first to the appropriate facilities and/or personnel). Using a step-wise collection of triage data, triage bot nodes 809 and triage management system 807 can work together to not only sort and prioritize patients and patient data, they can likewise sort and prioritize (D) the dissemination of information about patients in a better coordinated response to an emergency. As response to the event progresses, group management system 810 may add user nodes to a group or may re-assign user nodes to new and/or additional groups to optimize coverage, responsiveness, medical expertise, event response management, etc. This dynamic modification of triage node groups can be partially or wholly automated, but also may be controlled in whole or in part by human managers who are on-site and/or located remotely (e.g., in hospitals or other strategic locations).

Some implementations also include triage bot nodes 809 and/or other components of system 800 that are more actively involved in the triage process. For example, if a given community, governmental agency or other grouping utilizes a specific triage plan that uses specific types of patient data, components of system 800 can ensure that such patient data is collected promptly, in a desired sequence, and/or that the data is checked for any obvious errors prior to being used for prioritization of patient treatment and other responses. Other functions may be served by triage bot nodes 809 and/or triage management system 807. However, as a non-limiting example, each triage bot node 809 may be equipped and/or configured to lead a user through a pre-scripted protocol or data collection process that ensures the collection and verification of minimum patient data that permits uniform and consistent triaging of patients in the emergency response. Moreover, triage bot node 809 can be configured to ask additional questions to prompt a user to examine the patient for other injuries, etc. (e.g., burns, skin discoloration, lacerations, etc.). This pre-scripted checklist and sequencing of patient data collection questions provides a better organized and more efficient patient data collection process for the entire emergency scene without requiring the responders to have to remember all of the issues to be addressed or to have to consult documents and/or other materials in the midst of their response work.

A non-limiting example of bot support in triage group communications includes receipt of data relating to specific symptoms, which can also can trigger a data collection protocol for determining whether or not a victim/patient is suffering from a specific type of condition, injury, disease, etc. In one such non-limiting example, a triage group user node might report two or more symptoms consistent with a patient being in shock (e.g., rapid pulse and cool, clammy skin). Upon receiving this information, a triage bot node 809, triage management system 807, facility 834, and/or other segments of system 800 can call up a shock question script/list that is delivered to a user node 802 to determine whether other symptoms of shock are present (e.g., pale/ashen skin, nausea, pupil enlargement, fatigue, etc.). Thus triage via a user node 802 can be performed more effectively with reduced risk of omission or failure to recognize a given condition, etc. Moreover, a triage management system 807 or the like can track trends in the condition of many patients across time and geography so that common conditions, symptoms, etc. can be correlated to suggest a given situation (e.g., widespread, common symptoms of chlorine gas poisoning such as sneezing, throat/nose irritation, eye irritation, conjunctivitis, etc.).

When user nodes include implementations such as those discussed herein using a two-device node composition (e.g., an intermediate communication device paired with an end user device, for example using Bluetooth LE), additional advantages can be realized in emergency support utilizing group communications for triaging. For example, if an end user device is a push-to-talk device as described herein, then a responder may only have to talk into the end user device to supply patient triage data to a triage bot node 809 and/or triage management system 807. Likewise, if a user node further includes an intermediate communication device (e.g., a smartphone, cellphone, tablet or other highly portable device), then that device may also be used as part of the patient data collection apparatus. For example, a smartphone camera and microphone can be used to provide photographic and acoustic supporting data regarding a patient's condition and/or conditions in the vicinity of the emergency.

As user nodes 802 collect triage data (which can include patient data and possibly other relevant data to assist in triaging the emergency) sent as voice data by users, the collected data is sent to a group's triage bot node 809. In some implementations each triage bot node 809 converts the voice data into text and/or other data formats that can be evaluated for content and processed. In other implementations the conversion from voice to another data format can be performed by the triage management system 807 (e.g., in a manner similar to that shown in connection with group management service 310 in FIG. 4). Processing of the converted voice data can include sorting the relevant patient data into triage categories. In some implementations the patient data also can be used as inputs to triage evaluation algorithms or other processes that permit the prioritization of patient care and, in some implementations, the assignment of patients to various facilities and/or treatment locations that permit the efficient utilization of resources and personnel to improve patient care allocation. Where multiple groups each have a triage bot node 809 performing this type of collection, supplementation, conversion and sorting of patient data and any other triage data, the multiple triage bot nodes 809 can then forward their preliminary triage results to the triage management system 807 for master sorting and prioritization, as well as auditing, recordkeeping and overall triage management of the emergency situation. A triage management system 807 may also make recommendations to individual groups as to deployment of triage personnel and other resources based on data collected from dispatchers, group management system 810 and/or any other source of triage service allocation information. By collecting triage data from multiple locations simultaneously, a triage management system 807 also can notify, coordinate with and make assignments of patients to (E) facilities (e.g., facilities 830, 832, 834 of FIG. 8) based on optimizing resources, expertise, etc. at available facilities and with available transportation. In implementations where the “raw” collected patient data is sent immediately to the triage management system 807 without prioritization by the triage bot nodes 809, the triage management system 807 can perform such prioritization and send notifications (E) to the groups 821, 822, 823 with additional instructions, field treatment and/or triage recommendations, etc.

As triage data is collected, triage management system 807 and/or group management system 810 may determine that changing conditions and/or updated information indicate that a change in triage group composition(s) require modification. For example, if specific medical conditions are found (e.g., the presence of a poisoning agent, radiation, etc.), additional personnel capable of assisting in the triage may be deployed to the incident location, or may simply be “re-assigned” to different triage groups that are in need of certain skills or knowledge. Similarly, such updated information can lead to dropping a group member user node and re-assigning that individual to a different group. Such dynamic modification of groups based on changing conditions and/or knowledge permit rapid adaptation of triage group and personnel deployments based on centralized coordination of the triage operation based on collected triage data from the triage groups (e.g., groups 821, 822, 823). Artificial intelligence (AI) can be employed (e.g., as part of software or one or more processes or applications (e.g. 840 of FIG. 8, or 900 of FIG. 9), which can be executed on one or more computing systems 852, 808 in system 800 of FIG. 8) to obtain voice-driven data and to determine whether steps and/or processes should be implemented, as well as monitoring other conditions, situations, etc. in the triage process.

Patients transported from the emergency event location(s) may then be treated at appropriate facilities (e.g., facilities 830, 832, 834). By using patient identification data that uniquely identifies each patient being triaged and may be supplemented by appropriate personnel (in some implementations by both members and non-member of a given triage group), the preliminary triage data collected for each patient may “follow” the patient to the treatment facility and be utilized by medical and other personnel in providing a seamless continuity of care and treatment.

Use of bot support in triage group communications can help in avoiding or minimizing “under-triage” (i.e., underestimating patient condition severity) and “over-triage” (i.e., overestimating patient condition severity), especially where different agencies are responding to an emergency and where individuals with varying degrees of training and experience may be involved in performing triage. Similarly, use of a triage management system 807 and standardized triage bot node 809 capabilities can improve the consistency and reliability of triaging performed in different settings and in different conditions. Similarly, having centralized triage clearing and evaluation can facilitate and help in the coordination of patient evacuation from the scene of the emergency.

In some implementations the software or other analysis tools used in connection with the triage bot nodes and/or triage management system may be configured to analyze the triage data to detect patient conditions and the like that might not be readily apparent to a responder performing triage. Such notification of a potential latent or non-obvious condition or problem can allow the responder and/or other personnel to proactively evaluate the patient for such a condition or problem earlier than would possible with traditional triaging techniques and approaches.

Referring to FIG. 9, a method 900 of providing bot support in triage group communications in a distributed group communication environment is shown. Method 900 (which may be the same or similar to software or application 840 of FIG. 8 in some implementations) is described with reference to non-limiting exemplary elements, apparatus, systems and other information from FIG. 8 and other Figures in some implementations. The description below references operations of FIG. 9 parenthetically.

As described in connection with FIG. 8, an event occurs and a call for assistance is made in response to an event (905), for example through calls, alarms, alerts and other communications sent to and from authorities and the like. An event identification message is then received (910) by a group management system, which responsively creates (915) one or more triage node groups that respond to the event notification, where each triage group comprises team members (e.g., user nodes and at least one triage bot node) who can exchange voice messages and send voice messages to the triage bot node (917) that include patient data and/or other triage data in the course of responding to the identified event. Some of these messages may be part of a pre-scripted patient data protocol/collection process and/or review/confirmation of potential symptoms and the like (919).

Each triage bot node in a group collects and then (920) organizes, prioritizes, etc. patient data and/or other triage data. If data suggests that substantial danger is imminent or time-critical response is needed, then the triage bot node in a given group can respond to address the detected situation. Each triage bot node may forward (923) patient data and/or other triage data collected from group members to a triage management system that is configured to coordinate patient transportation, treatment, etc. for the reported incident.

The triage management system organizes, prioritizes (925) and generates responses to the patient data and other triage data it receives. The generated responses based on the patient data and other triage data can include (930) issuing patient transportation and treatment instructions to triage and other incident response team members and the facilities that will be handling patients.

Throughout process 900 a log may be generated (935) of triage group member communications, messages, etc., as well as instructions and other communications issued by the group management system and triage management system. Based on triage data collected and evaluated by triage bot nodes and the triage management system, dynamic modification (940) of the triage node groups' memberships can be performed throughout process 900 (e.g., adding new members to various groups, combining groups, adding specialists as users of user nodes, linking/adding a user node based at a hospital or other location for a specific group, etc.).

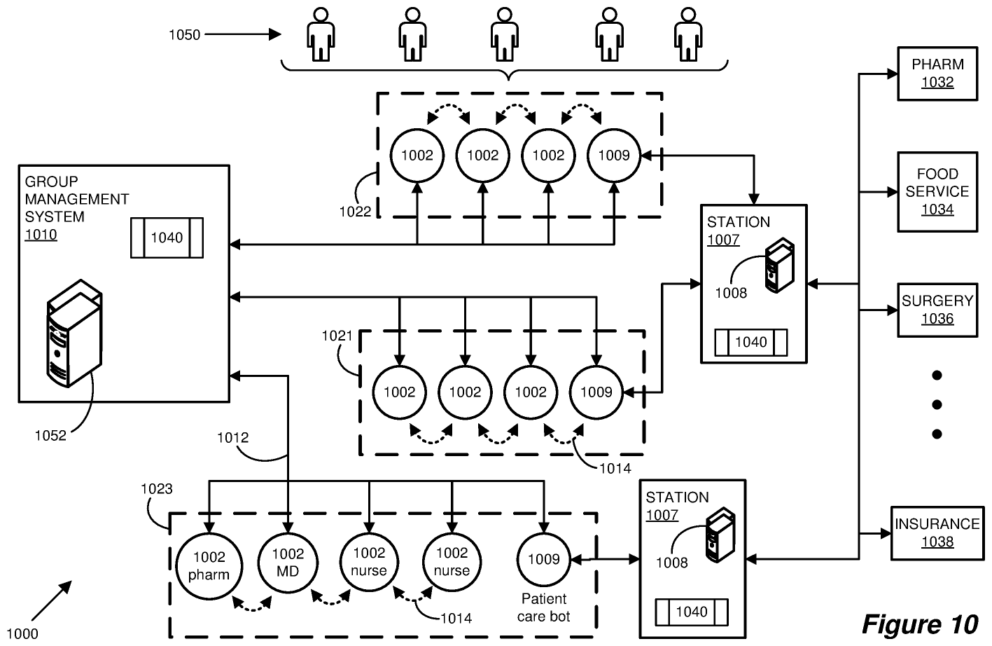

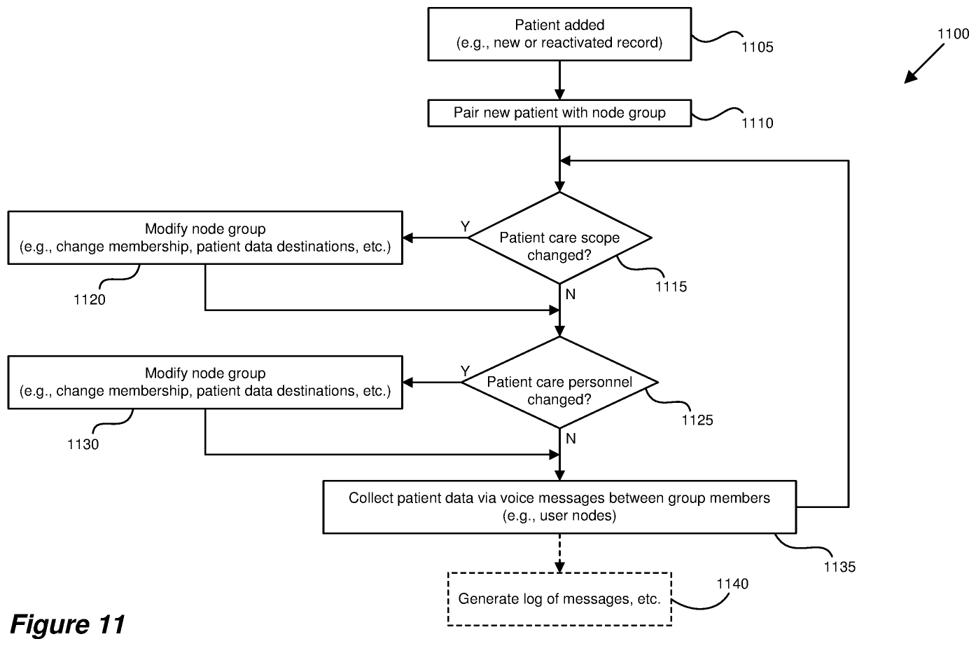

In many medical evaluation and treatment settings, implementations of bot support in patient care group communications can be used to perform patient care (e.g., recordkeeping, medication conflict checking, surgery preparation, billing, etc.)—during, after and in addition to an emergency support setting alone. The one or more exemplary systems illustrated in FIG. 10 show implementations of bot support in patient care group communications. In the non-limiting example of FIG. 10, multiple patient care groups 1021, 1022, 1023 can be created by a group management system 1010 (e.g., a computing system comprising one or more servers 1052 or the like and running software or an application 1040, which may in some implementations be the same or similar to process 1100 of FIG. 11). Patient care node groups 1021-1023 can each include one or more patient care team user nodes 1002 and a patient care record bot node 1009. Nodes 1002, 1009 of each group 1021-1023 may communicate with one another via other appropriate links 1012, 1014 either via group management system 1010 or directly with one another. Patient care team user nodes 1002 are configured to allow users to send messages between and among the members of each communication group 1021-1023 during the groups' care of one or more patients. These messages are received, monitored and evaluated by patient care record bot nodes 1009. Operation of the user nodes, bot nodes and node groups can be the same or similar to those of the triage systems described herein in some implementations.

In some implementations, the non-limiting exemplary system of FIG. 10 can be scaled to include more communication groups managed by group management system 1010. Also, there may be multiple groups managed by multiple group management systems similar to system 1010, where these group management systems coordinate their patient care record data flows to appropriate locations (e.g., departments, floors, or other divisions within a hospital or other facility, even across multiple facilities such as veterans facilities, etc.). Moreover, the system 1000 of FIG. 10 may be “reverse scaled” so that only one communication group is created by group management system 1010, but the single created group has many user nodes 1002 that can communicate with one or more patient care record bot nodes (that might also have membership in the single communication group). Other topographies and organizational schemes can be implemented as well.

Assignment of a node group 1022 to a given patient or a patient set can be based on one or more various factors. One non-limiting example is to assign patients to a group 1022 based on the type of care the patients are receiving—all patients in maternity care may be assigned to one group, while a different node group 1022 has pediatric patients assigned to it. Similarly, patients in different facilities or locations may be assigned to a common communication node group based on various factors. Each node group 1022 can likewise be connected to a central communication or monitor station 1007 that is related to the type of care being administered to the patients affiliated with the group 1022. Station 1007 can be a nurses station, monitoring station or other type of location (e.g., a computing system comprising one or more servers 1008 or the like and running software or an application 1040, which may in some implementations be the same or similar to process 1100 of FIG. 11). In other implementations a node group may be assigned to each patient. The group members can then provide seamless care and recordation of patient data. A group management system 1010 may dynamically modify each group's composition based on factors such as expanded patient care scope, personnel transitions (e.g., doctors, nurses, others going on and off shift, new staff being assigned to patient care, etc.), patient transfer between facilities, etc.

In one non-limiting example, one of the communication node groups 1022 has users who are assigned to care for a group of patients 1050. The user members of group 1022 can be physicians, nurses, physical therapists, pharmacists, dieticians and others. In some situations, a given user (e.g., a pharmacist) may be a member of multiple groups whose different patient groups are serviced by the pharmacist. Each patient in patient group 1050 is assigned a unique identifier (e.g., a patient number, a bar code, a name (so long as it is unique within the appropriate group or other set of patient identifiers), a room number or some other identifier, including a combination of identifiers). As users perform patient care services for a given patient, the patient care record bot acting as a bot node 1009 for a given communication group records the information pertaining to such care. Recordation can include voice-to-text conversion for reporting, auditing and other functions. In some implementations a gateway processing system, such as the non-limiting example of system 311 of FIG. 4, can be utilized to facilitate bot node evaluation of message content and redirection of message content and/or other communications to the appropriate external entity, station, system, etc. that can assist one or more of groups 1021-1023 (which may communicate with one another via other appropriate links 1012, 1014 either via group management system 1010 or directly with one another) and further assist group management system 1010 and/or a (virtual or actual) facilities station 1007 in the dynamic formation and modification of one or more patient care node groups. Artificial intelligence (AI) can be employed (e.g., as part of software or one or more processes or applications (e.g. 1040 of FIG. 10, or 1100 of FIG. 11), which can be executed on one or more computing systems 1052, 1008 in system 1000 of FIG. 10) to obtain voice-driven data and to determine whether conflicting patient care steps and/or processes are being provided, as well as monitoring drug use by the patients and other safety and patient care procedures. For example, if a patient is due for a diagnostic procedure that requires fasting or abstaining from certain medications for a prescribed antecedent period, that restriction can be enforced and/or broadcast by system 1010 or a station 1007 if a conflicting instruction or request is received via voice-implemented patient data.

As data is collected for a given patient, the data can be stored (after sorting and/or editing, if appropriate) for multiple uses. As seen in the non-limiting examples of FIG. 10, processed patient care data can be directed to various databases, recordkeeping locations and the like. In FIG. 10 patient data generated by the patient care record groups 1022 is sent by each group's patient care record bot 1009 and can be stored or otherwise made available to pharmacy records, insurance (e.g., for billing and/or other purposes), surgery scheduling, patient food service, etc. In some implementations patient care notifications can be sent to the pharmacy 1032, food service 1034, surgery scheduling 1036, insurance 1038 and/or other patient care segments based on collected patient care data. In one non-limiting example, medication conflict checking can be performed based on patient data obtained from voice messages sent by patient care user nodes.

Referring to FIG. 11, a method 1100 of providing bot support in patient care group communications for coordinated patient care in a distributed group communication environment is shown. Method 1100 (which may be the same or similar to software or application 1040 of FIG. 10 in some implementations) is described with reference to exemplary elements, apparatus, systems and other information from Figure 10 and other Figures in some implementations. The description below references operations of FIG. 11 parenthetically.

As described in connection with FIG. 10, when a patient is added to a recordkeeping or other system (or perhaps has an existing historical record that is “reactivated” upon a new medical condition being treated or some other triggering event), the addition of the new patient (1105) is noted (e.g., by group management system 1010 or the like). The patient and a patient care node group are paired (1110)—for example, the new patient may be added to an existing node group or a node group may be created or updated to manage the records and patient data of the new patient. As the patient's care progresses, changes may take place to the scope of the patient's care (1115). When such changes necessitate or otherwise lead to a modification to the node group caring for that patient, then an appropriate node group dynamic modification is made (1120). Similarly, if there are changes in patient care personnel or the like regarding the patient's care (1125), then an appropriate node group dynamic modification is made (1130). With the appropriate node group identified and connected to a patient care bot node 1009, patient data is collected (e.g., via voice messages that are sent either between node group members or directly to patient care bot node 1009). A log of messages and/or other data pertaining to patient care delivered by a particular node group or the like may be generated (1140) and updated as desired.

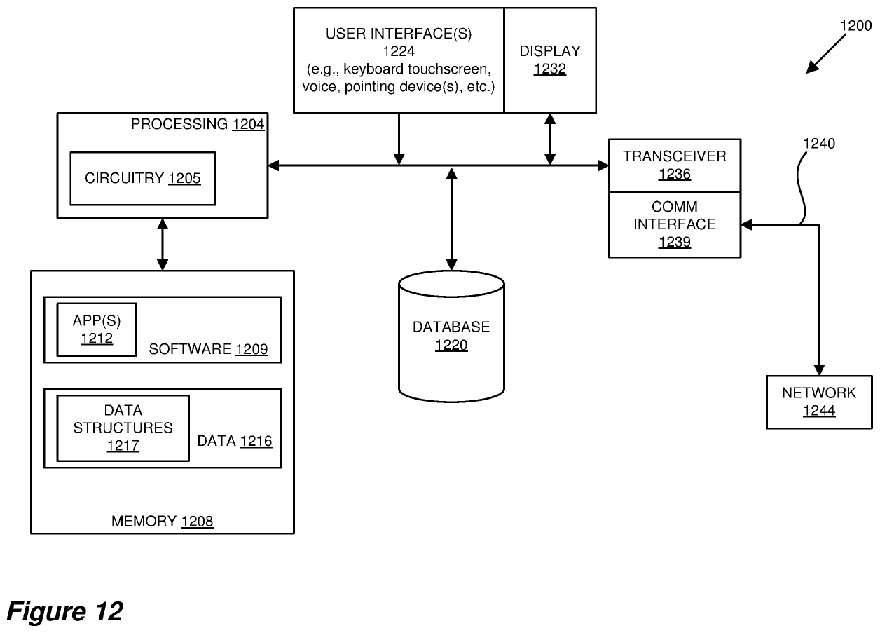

FIG. 12 illustrates a representative computer 1200 (e.g., a computing system, computing device, computing architecture, etc.) in accordance with implementations disclosed herein. Computer 1200 can perform processes, applications, software and other implementations of methods (non-limiting examples of which may include applications 135, 155 of FIG. 1, software 208 and/or application 288 of FIG. 2, an application running on a device 307 of FIG. 3A, software 340 of FIGS. 3B and 4, software/application 640 of FIG. 6, process 700 of FIG. 7, software/application 840 of FIG. 8, process 900 of FIG. 9, software/application 1040 of FIG. 10, and/or process 1100 of FIG. 11). Computer 1200 may be any sort of known computing device including servers, desktop computers, notebook computers, tablets, embedded computers, personal digital assistants (PDAs), smart phones, wearable computers, or any other sort of device or combination of devices.

Computer 1200 includes memory 1208, which may include one or both of volatile and nonvolatile memory types. In some embodiments, memory 1208 includes applications, software and/or firmware that includes program instructions that are fetched and executed, including program instructions for the methods, processes, software and/or applications described herein. Systems, methods and other implementations described herein may be provided in the form of tangible and non-transitory machine-readable medium or media (such as a hard disk drive, hardware memory, etc.) having instructions recorded thereon for execution by a computer and/or one or more processors, microprocessors and/or other circuitry that retrieves and executes the instructions. The set of instructions may include various commands that instruct the computer and/or one or more processors, microprocessors and/or other circuitry to perform specific operations such as the methods and processes of the various implementations described and/or disclosed herein. The set of instructions may be in the form of a software program or application. The computer storage media may include volatile and non-volatile media, and removable and non-removable media, for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic disk storage, or any other hardware medium which may be used to store desired information and that may be accessed by components of the system. Components of the system may communicate with each other via wired or wireless communication. The components may be separate from each other, or various combinations of components may be integrated together into a portable device (or combination of devices) or processor (or combination of processors), or contained within a workstation with standard computer hardware (for example, processors, circuitry, logic circuits, memory, and the like). The system may include processing devices such as microprocessors, microcontrollers, integrated circuits, control units, storage media, and other hardware.

Memory 1208 of FIG. 12 includes software 1209 (which may include one or more applications 1212) and data 1216 (which may include data structures 1217). Non-limiting examples of non-volatile memory include, but are not limited to, flash memory, SD, Erasable Programmable Read Only Memory (EPROM), Electrically Erasable Programmable Read Only Memory (EEPROM), hard disks, and Non-Volatile Read-Only Memory (NOVRAM). Non-limiting examples of volatile memory store various data structures and user data and include, but are not limited to, Static Random Access Memory (SRAM), Dual Data Rate Random Access Memory (DDR RAM), Dual Data Rate 2 Random Access Memory (DDR2 RAM), Dual Data Rate 3 Random Access Memory (DDR3 RAM), Zero Capacitor Random Access Memory (Z-RAM), Twin-Transistor Random Access Memory (TTRAM), Asynchronous Random Access Memory (A-RAM), ETA Random Access Memory (ETA RAM), and other forms of temporary memory.

In addition to memory 1208, computer 1200 may also include a database 1220, which may be local to computer 1200 or externally accessible to computer 1200 such as in a cloud environment providing cloud storage. Database 1220 may provide storage for a large number of parameters including data structures 1217, recorded or decoded audio from user node messages, voice libraries, and/or any other applications and/or data associated with implementations disclosed herein.

Computer 1200 includes a processing system 1204. Processing system 1204 includes one or more processing devices (e.g., circuitry 1205 and/or other components) suitable for executing applications 1212 and data 1216 such as Intel x86-compatible processors, embedded processors, mobile processors, and/or RISC processors. Processing system 1204 may include several devices including field-programmable gate arrays (FPGAs), memory controllers, North Bridge devices, and/or South Bridge devices. Although in most embodiments, processing system 1204 fetches application program instructions and data from memory 1208, it should be understood that processing system 1204 and applications 1212 may be configured in any allowable hardware/software configuration, including pure hardware configurations implemented in ASIC or FPGA forms.

Computer 1200 also may include one or more user interfaces such as a display 1232 in some implementations. The display 1232 may include control and non-control areas. In most embodiments, controls are “soft controls” displayed on a screen and not necessarily hardware controls or buttons. In some embodiments one or more controls may be “soft controls” and one or more controls may be hardware controls or buttons. In yet other embodiments, controls may be all hardware controls or buttons. Display 1232 may be a touchscreen whereby controls may be activated by a finger touch or touching with a stylus or pen. Non-control areas are areas of the screen not including a control. Computer 1200 may also include one or more additional user interfaces 1224 (e.g., a keyboard, a microphone (e.g., configured to receive audio instructions, etc.), a speaker, a pointing device, etc.) to interact with applications and any display 1232.

Computer 1200 may include a transceiver 1236 (e.g., a network transceiver), which can be a wired or wireless interface able to connect to one or more networks, including the Internet or cloud in order to transmit and receive messages, management instructions, etc. Transceiver 1236 may also be configured to transmit and receive audio data, text, enhanced text, and other types of communications. In some implementations the transceiver 1236 may operate via a network interface 1239 to connect to a network 1244 via one or more wired and/or wireless connections 1240.

The descriptions and figures included herein depict specific implementations to teach those skilled in the art how to make and use the best option. For the purpose of teaching inventive principles, some conventional aspects have been simplified or omitted. Those skilled in the art will appreciate variations from these implementations that fall within the scope of the disclosure. Those skilled in the art will also appreciate that the features described above can be combined in various ways to form multiple implementations. Moreover, some features, processes, etc. of one implementation (e.g., bot support in triage group communications) may be utilized in connection with another implementation (e.g., bot support in emergency services group communications or bot support in patient care group communications), and thus the teachings of one application may be applicable to other applications as well. As a result, the invention is not limited to the specific implementations described above, but only by the claims and their equivalents.

Example embodiments are disclosed below:

1. A method of managing a communications group, the method comprising:
receiving an event identification message identifying an event requiring triage assistance;
creating:
a first triage node group comprising a first plurality of triage node group communication nodes, wherein the first plurality of triage node group communication nodes comprises a first plurality of triage node group user nodes and a first triage bot node, wherein the first plurality of triage node group user nodes is configured to receive first patient data and forward the received first patient data to the first triage bot node, further wherein the first triage bot node is configured to generate first triage node group data based on the received first patient data;
a second triage node group comprising a second plurality of triage node group communication nodes, wherein the second plurality of triage node group communication nodes comprises a second plurality of triage node group user nodes and a second triage bot node, wherein the second plurality of triage node group user nodes is configured to receive second patient data and forward the received second patient data to the second triage bot node, further wherein the second triage bot node is configured to generate second triage node group data based on the received second patient data;
prioritizing the first triage node group data and second triage node group data to generate prioritized event data.

2. The method of claim 1 wherein generating the first triage node group data comprises the first triage bot node prioritizing the first patient data to generate prioritized first patient data; and
further wherein generating the second triage node group data comprises the second triage bot node prioritizing the second patient data to generate prioritized second patient data.

3. The method of claim 1 wherein a first triage node group user node of the first plurality of triage node group user nodes comprises a two-device node comprising an intermediate communication device paired with a voice unit, wherein the intermediate communication device is connected to a group communication network.

4. The method of claim 3 wherein the intermediate communication device is paired to the voice unit using Bluetooth LE.

5. The method of claim 1 wherein a first triage node group user node of the first of triage node group user nodes comprises a stand-alone device connected to a group communication network.

6. The method of claim 1 further comprising issuing patient transportation and treatment notifications to one or more healthcare facilities based on the prioritized event data.

7. The method of claim 6 wherein the steps are performed by a group management system and a triage management system operating in concert as either a single system or as two coordinated systems.

8. The method of claim 1 wherein each triage bot node is configured to lead a user node user through a pre-scripted protocol or data collection process.

9. The method of claim 1 wherein each triage bot node is configured to pose questions to a user node user to lead the user through an examination of a triage patient.

10. A non-transitory computer readable storage medium configured to store computer instructions that when executed cause one or more processors to perform:
receiving an event identification message identifying an event requiring triage assistance;
creating:
a first triage node group comprising a first plurality of triage node group communication nodes, wherein the first plurality of triage node group communication nodes comprises a first plurality of triage node group user nodes and a first triage bot node, wherein the first plurality of triage node group user nodes is configured to receive first patient data and forward the received first patient data to the first triage bot node, further wherein the first triage bot node is configured to generate first triage node group data based on the received first patient data;
a second triage node group comprising a second plurality of triage node group communication nodes, wherein the second plurality of triage node group communication nodes comprises a second plurality of triage node group user nodes and a second triage bot node, wherein the second plurality of triage node group user nodes is configured to receive second patient data and forward the received second patient data to the second triage bot node, further wherein the second triage bot node is configured to generate second triage node group data based on the received second patient data;
prioritizing the first triage node group data and second triage node group data to generate prioritized event data.

11. The non-transitory computer readable storage medium of claim 10 wherein generating the first triage node group data comprises the first triage bot node prioritizing the first patient data to generate prioritized first patient data; and further wherein generating the second triage node group data comprises the second triage bot node prioritizing the second patient data to generate prioritized second patient data.

12. The non-transitory computer readable storage medium of claim 10 wherein a first triage node group user node of the first of triage node group user nodes comprises a two-device node comprising an intermediate communication device paired with a voice unit, wherein the intermediate communication device is connected to a group communication network.

13. The non-transitory computer readable storage medium of claim 12 wherein the intermediate communication device is paired to the voice unit using Bluetooth LE.

14. The non-transitory computer readable storage medium of claim 10 wherein a first triage node group user node of the first of triage node group user nodes comprises a stand-alone device connected to a group communication network.

15. The non-transitory computer readable storage medium of claim 10 further configured to store computer instructions that when executed cause one or more processors to issue patient transportation and treatment notifications to one or more healthcare facilities based on the prioritized event data.

16. The non-transitory computer readable storage medium of claim 10 wherein each triage bot node is configured to lead a user node user through a pre-scripted protocol, a data collection process, and/or a series of questions configured to lead the user through an examination of a triage patient.

17. A method of managing a communications group, the method comprising:
receiving an event identification message identifying an event requiring triage assistance;
creating:
a first triage node group comprising a first plurality of triage node group communication nodes, wherein the first plurality of triage node group communication nodes comprises a first plurality of triage node group user nodes and a first triage bot node, wherein the first plurality of triage node group user nodes is configured to receive first patient data and forward the received first patient data to the first triage bot node, further wherein the first triage bot node is configured to generate first triage node group data based on the received first patient data;
a second triage node group comprising a second plurality of triage node group communication nodes, wherein the second plurality of triage node group communication nodes comprises a second plurality of triage node group user nodes and a second triage bot node, wherein the second plurality of triage node group user nodes is configured to receive second patient data and forward the received second patient data to the second triage bot node, further wherein the second triage bot node is configured to generate second triage node group data based on the received second patient data;
prioritizing the first triage node group data and second triage node group data to generate prioritized event data; and
dynamically modifying the first triage node group by adding a user node, removing a user node, adding a specialized triage bot node.

18. The method of claim 17 wherein each triage bot node is configured to lead a user node user through a pre-scripted protocol, a data collection process, and/or a series of questions configured to lead the user through an examination of a triage patient.

19. The method of claim 17 wherein a first triage node group user node of the first of triage node group user nodes comprises a two-device node comprising an intermediate communication device paired with a voice unit, wherein the intermediate communication device is connected to a group communication network and further wherein the intermediate communication device is paired to the voice unit using a wireless communication protocol.

20. The method of claim 17 wherein a first triage node group user node of the first of triage node group user nodes comprises a stand-alone device connected to a group communication network.

21. A method of managing a communications group, the method comprising:
receiving patient identification data;
creating a patient care record based on the received patient identification data;
pairing the patient care record with a patient care node group, wherein the patient care node group comprises a plurality of patient care user nodes and a patient care bot node;
collecting patient data via voice messages generated by the plurality of patient care user nodes; and
dynamically modifying the patient care node group based on one or more of the following:
a change in patient care scope;
a change in patient location;
a change in patient care personnel; and/or
patient data obtained from voice messages sent by the plurality of patient care user nodes.

22. The method of claim 21 further comprising generating a log of patient care user node messages and patient care node group modifications.

23. The method of claim 21 wherein a first patient care user node of the plurality of patient care user nodes comprises a two-device node comprising an intermediate communication device paired with a voice unit, wherein the intermediate communication device is connected to a group communication network.

24. The method of claim 23 wherein the intermediate communication device is paired to the voice unit using Bluetooth LE.

25. The method of claim 21 wherein a first patient care user node of the plurality of patient care user nodes comprises a stand-alone device connected to a group communication network.

26. The method of claim 21 further comprising medication conflict checking based on patient data obtained from voice messages sent by the plurality of patient care user nodes.

27. The method of claim 21 wherein each patient care bot node is configured to pose questions to a patient care user node user to lead the user through patient-related process, a pre-scripted protocol, and/or a data collection process.

28. A non-transitory computer readable storage medium configured to store computer instructions that when executed cause one or more processors to perform:
receiving patient identification data;
creating a patient care record based on the received patient identification data;
pairing the patient care record with a patient care node group, wherein the patient care node group comprises a plurality of patient care user nodes and a patient care bot node;
collecting patient data via voice messages generated by the plurality of patient care user nodes; and
dynamically modifying the patient care node group based on one or more of the following:
a change in patient care scope;
a change in patient location;
a change in patient care personnel; and/or
patient data obtained from voice messages sent by the plurality of patient care user nodes.

29. The non-transitory computer readable storage medium of claim 28 further configured to store computer instructions that when executed cause one or more processors to generate a log of patient care user node messages and patient care node group modifications.

30. The non-transitory computer readable storage medium of claim 28 wherein a first patient care user node of the plurality of patient care user nodes comprises a two-device node comprising an intermediate communication device paired with a voice unit, wherein the intermediate communication device is connected to a group communication network.

31. The non-transitory computer readable storage medium of claim 30 wherein the intermediate communication device is paired to the voice unit using Bluetooth LE.

32. The non-transitory computer readable storage medium of claim 28 wherein a first patient care user node of the plurality of patient care user nodes comprises a stand-alone device connected to a group communication network.

33. The non-transitory computer readable storage medium of claim 28 further configured to store computer instructions that when executed cause one or more processors to perform a medication conflict check based on patient data obtained from voice messages sent by the plurality of patient care user nodes.

34. The non-transitory computer readable storage medium of claim 28 wherein each patient care bot node is configured to pose questions to a patient care user node user to lead the user through patient-related process, a pre-scripted protocol, and/or a data collection process.

35. A method of managing a communications group, the method comprising:
receiving patient identification data;
creating a patient care record based on the received patient identification data;
pairing the patient care record with a patient care node group, wherein the patient care node group comprises a plurality of patient care user nodes and a patient care bot node;
collecting patient data via voice messages generated by the plurality of patient care user nodes; and
detecting a change in one of the following based on the collected patient data;
patient care scope;
patient location; and/or
patient care personnel;
dynamically modifying the patient care node group based on the detected change.

36. The method of claim 35 further comprising sending a patient care notification based on collected patient care data to one of the following:
a pharmacy;
patient care food service;
surgery scheduling;
insurance.

37. The method of claim 35 wherein at least one patient care user node of plurality of patient care user nodes comprises a two-device node comprising an intermediate communication device paired with a voice unit.

38. The method of claim 37 wherein the intermediate communication device is connected to a group communication network.

39. The method of claim 38 wherein intermediate communication device is paired to the voice unit using a wireless communication protocol.

40. The method of claim 35 wherein at least one patient care user node of plurality of patient care user nodes comprises a stand-alone device connected to a group communication network.

41. A method of managing a communications group, the method comprising:
receiving an event identification message identifying an event requiring emergency response assistance;
creating a response node group comprising a plurality of response node group communication nodes, wherein the plurality of response node group communication nodes comprises a first response node group user node and a risk identification node, wherein the risk identification node comprises a risk identification bot node and/or a risk identification user node, wherein any bot node comprises a software application configured to perform one or more tasks over the Internet;
evaluating response node group user node voice message content;
identifying a risk based on the evaluated response node group user node voice message content;
modifying the response node group, wherein modifying the response node group comprises adding a risk assessment node to the response node group, wherein the risk assessment node comprises a risk assessment bot node and/or a risk assessment user node; and
providing information and/or recommended actions to the first response team user node.

42. The method of claim 41 wherein the first response node group user node comprises a two-device node comprising an intermediate communication device paired with a voice unit, wherein the intermediate communication device is connected to a group communication network.

43. The method of claim 42 wherein the intermediate communication device is paired to the voice unit using Bluetooth LE.

44. The method of claim 41 wherein the first response node group user node comprises a stand-alone device connected to a group communication network.

45. The method of claim 41 wherein evaluating response node group user node voice message content comprises converting voice message audio data to text data and evaluating text data content.

46. The method of claim 41 wherein the risk assessment node comprises a risk assessment bot node and further wherein the risk assessment bot node is accessed at or obtained from a bot library.

47. The method of claim 46 wherein the risk assessment bot node is configured to pose questions to one or more response node group user nodes to request additional information to assist in evaluating the identified risk and/or response node group user node voice message audio content.

48. The method of claim 41 further comprising dynamically modifying user node membership of the response node group based on risk assessment information collected from the response node group member nodes.

49. A non-transitory computer readable storage medium configured to store computer instructions that when executed cause one or more processors to perform:
receiving an event identification message identifying an event requiring emergency response assistance;
creating a response node group comprising a plurality of response node group communication nodes, wherein the plurality of response node group communication nodes comprises a first response node group user node, a second response node group user node, and a risk identification bot node, wherein the risk identification bot node comprises a software application configured to perform one or more tasks over the Internet; and
adding a risk assessment bot node to the response node group, wherein the risk assessment bot node comprises a software application configured to perform one or more tasks over the Internet; and
wherein the risk identification node is configured to evaluate response node group user node voice message content and to identify a risk based on the evaluated response node group user node voice message content;
further wherein the risk assessment node is selected based on a risk identified by the risk identification bot node and is configured to provide information and/or recommended actions to the first and second response team user nodes based on evaluated response node group user node voice message content.

50. The non-transitory computer readable storage medium of claim 49 wherein at least one of the first and second response node group user nodes comprises a two-device node comprising an intermediate communication device paired with a voice unit, wherein the intermediate communication device is connected to a group communication network.

51. The non-transitory computer readable storage medium of claim 50 wherein the intermediate communication device is paired to the voice unit using Bluetooth LE.

52. The non-transitory computer readable storage medium of claim 49 wherein at least one of the first and second response node group user nodes comprises a stand-alone device connected to a group communication network.

53. The non-transitory computer readable storage medium of claim 49 wherein the risk identification node evaluating response node group user node voice message content comprises converting voice message audio data to text data and evaluating text data content.

54. The non-transitory computer readable storage medium of claim 49 wherein the risk assessment bot node is accessed at or obtained from a bot library.

55. The non-transitory computer readable storage medium of claim 49 further comprising the group management system dynamically modifying user node membership of the response node group based on risk assessment information collected from the response node group member nodes.

56. A method of managing a communications group, the method comprising:
a group management system receiving an event identification message identifying an event requiring emergency response assistance;
the group management system creating a response node group comprising a first response node group user node, a second response node group user node, and a risk identification bot node, wherein the risk identification bot node comprises a risk identification software application configured to perform one or more tasks over the Internet, wherein the risk identification bot node is configured to evaluate response node group user node voice message audio content;
identifying a risk based on the evaluated response node group user node voice message audio content;
adding a risk assessment bot node to the response node group, wherein the risk assessment bot node is configured to assist in evaluating a risk identified by the risk identification bot node; and
the risk assessment bot node providing information and/or recommended actions to the first and second response team user nodes based on evaluation of response node group user node voice message audio content.

57. The method of claim 56 wherein at least one of the first and second response node group user nodes comprises a two-device node comprising an intermediate communication device paired with a voice unit, wherein the intermediate communication device is connected to a group communication network and further wherein the intermediate communication device is paired to the voice unit using a wireless communication protocol.

58. The method of claim 57 wherein at least one of the first and second response node group user nodes comprises a stand-alone device connected to a group communication network.

59. The method of claim 56 wherein the risk assessment bot node is configured to pose questions to response node group user nodes to request additional information based on based on evaluation of response node group user node voice message audio content.

What is claimed is:

1. A method of managing a communications group, the method comprising:
receiving an event identification message identifying an event requiring triage assistance;
creating:
a first triage node group comprising a first plurality of triage node group communication nodes, wherein the first plurality of triage node group communication nodes comprises a first plurality of triage node group user nodes and a first triage bot node, wherein the first plurality of triage node group user nodes is configured to receive first patient data and forward the received first patient data to the first triage bot node, further wherein the first triage bot node is configured to generate first triage node group data based on the received first patient data;
a second triage node group comprising a second plurality of triage node group communication nodes, wherein the second plurality of triage node group communication nodes comprises a second plurality of triage node group user nodes and a second triage bot node, wherein the second plurality of triage node group user nodes is configured to receive second patient data and forward the received second patient data to the second triage bot node, further wherein the second triage bot node is configured to generate second triage node group data based on the received second patient data;
prioritizing the first triage node group data and second triage node group data to generate prioritized event data.

2. The method of claim 1 wherein generating the first triage node group data comprises the first triage bot node prioritizing the first patient data to generate prioritized first patient data; and
further wherein generating the second triage node group data comprises the second triage bot node prioritizing the second patient data to generate prioritized second patient data.

3. The method of claim 1 wherein a first triage node group user node of the first plurality of triage node group user nodes comprises a two-device node comprising an intermediate communication device paired with a voice unit, wherein the intermediate communication device is connected to a group communication network.

4. The method of claim 3 wherein the intermediate communication device is paired to the voice unit using Bluetooth LE.

5. The method of claim 1 wherein a first triage node group user node of the first of triage node group user nodes comprises a stand-alone device connected to a group communication network.

6. The method of claim 1 further comprising issuing patient transportation and treatment notifications to one or more healthcare facilities based on the prioritized event data.

7. The method of claim 6 wherein the steps are performed by a group management system and a triage management system operating in concert as either a single system or as two coordinated systems.

8. The method of claim 1 wherein each triage bot node is configured to lead a user node user through a pre-scripted protocol or data collection process.

9. The method of claim 1 wherein each triage bot node is configured to pose questions to a user node user to lead the user through an examination of a triage patient.

10. A non-transitory computer readable storage medium configured to store computer instructions that when executed cause one or more processors to perform:
receiving an event identification message identifying an event requiring triage assistance;
creating:
a first triage node group comprising a first plurality of triage node group communication nodes, wherein the first plurality of triage node group communication nodes comprises a first plurality of triage node group user nodes and a first triage bot node, wherein the first plurality of triage node group user nodes is configured to receive first patient data and forward the received first patient data to the first triage bot node, further wherein the first triage bot node is configured to generate first triage node group data based on the received first patient data;
a second triage node group comprising a second plurality of triage node group communication nodes, wherein the second plurality of triage node group communication nodes comprises a second plurality of triage node group user nodes and a second triage bot node, wherein the second plurality of triage node group user nodes is configured to receive second patient data and forward the received second patient data to the second triage bot node, further wherein the second triage bot node is configured to generate second triage node group data based on the received second patient data;
prioritizing the first triage node group data and second triage node group data to generate prioritized event data.

11. The non-transitory computer readable storage medium of claim 10 wherein generating the first triage node group data comprises the first triage bot node prioritizing the first patient data to generate prioritized first patient data; and
further wherein generating the second triage node group data comprises the second triage bot node prioritizing the second patient data to generate prioritized second patient data.

12. The non-transitory computer readable storage medium of claim 10 wherein a first triage node group user node of the first of triage node group user nodes comprises a two-device node comprising an intermediate communication device paired with a voice unit, wherein the intermediate communication device is connected to a group communication network.

13. The non-transitory computer readable storage medium of claim 12 wherein the intermediate communication device is paired to the voice unit using Bluetooth LE.

14. The non-transitory computer readable storage medium of claim 10 wherein a first triage node group user node of the first of triage node group user nodes comprises a stand-alone device connected to a group communication network.

15. The non-transitory computer readable storage medium of claim 10 further configured to store computer instructions that when executed cause one or more processors to issue patient transportation and treatment notifications to one or more healthcare facilities based on the prioritized event data.

16. The non-transitory computer readable storage medium of claim 10 wherein each triage bot node is configured to lead a user node user through a pre-scripted protocol, a data collection process, and/or a series of questions configured to lead the user through an examination of a triage patient.

17. A method of managing a communications group, the method comprising:
receiving an event identification message identifying an event requiring triage assistance;
creating:
a first triage node group comprising a first plurality of triage node group communication nodes, wherein the first plurality of triage node group communication nodes comprises a first plurality of triage node group user nodes and a first triage bot node, wherein the first plurality of triage node group user nodes is configured to receive first patient data and forward the received first patient data to the first triage bot node, further wherein the first triage bot node is configured to generate first triage node group data based on the received first patient data;
a second triage node group comprising a second plurality of triage node group communication nodes, wherein the second plurality of triage node group communication nodes comprises a second plurality of triage node group user nodes and a second triage bot node, wherein the second plurality of triage node group user nodes is configured to receive second patient data and forward the received second patient data to the second triage bot node, further wherein the second triage bot node is configured to generate second triage node group data based on the received second patient data;
prioritizing the first triage node group data and second triage node group data to generate prioritized event data; and
dynamically modifying the first triage node group by adding a user node, removing a user node, adding a specialized triage bot node.

18. The method of claim 17 wherein each triage bot node is configured to lead a user node user through a pre-scripted protocol, a data collection process, and/or a series of questions configured to lead the user through an examination of a triage patient.

19. The method of claim 17 wherein a first triage node group user node of the first of triage node group user nodes comprises a two-device node comprising an intermediate communication device paired with a voice unit, wherein the intermediate communication device is connected to a group communication network and further wherein the intermediate communication device is paired to the voice unit using a wireless communication protocol.

20. The method of claim 17 wherein a first triage node group user node of the first of triage node group user nodes comprises a stand-alone device connected to a group communication network.

21. A method of managing a communications group, the method comprising:
receiving patient identification data;
creating a patient care record based on the received patient identification data;
pairing the patient care record with a patient care node group, wherein the patient care node group comprises a plurality of patient care user nodes and a patient care bot node;
collecting patient data via voice messages generated by the plurality of patient care user nodes; and
dynamically modifying the patient care node group based on one or more of the following:
a change in patient care scope;
a change in patient location;
a change in patient care personnel; and/or
patient data obtained from voice messages sent by the plurality of patient care user nodes.

22. The method of claim 21 further comprising generating a log of patient care user node messages and patient care node group modifications.

23. The method of claim 21 wherein a first patient care user node of the plurality of patient care user nodes comprises a two-device node comprising an intermediate communication device paired with a voice unit, wherein the intermediate communication device is connected to a group communication network.

24. The method of claim 23 wherein the intermediate communication device is paired to the voice unit using Bluetooth LE.

25. The method of claim 21 wherein a first patient care user node of the plurality of patient care user nodes comprises a stand-alone device connected to a group communication network.

26. The method of claim 21 further comprising medication conflict checking based on patient data obtained from voice messages sent by the plurality of patient care user nodes.

27. The method of claim 21 wherein each patient care bot node is configured to pose questions to a patient care user node user to lead the user through patient-related process, a pre-scripted protocol, and/or a data collection process.

28. A non-transitory computer readable storage medium configured to store computer instructions that when executed cause one or more processors to perform:
receiving patient identification data;
creating a patient care record based on the received patient identification data;
pairing the patient care record with a patient care node group, wherein the patient care node group comprises a plurality of patient care user nodes and a patient care bot node;
collecting patient data via voice messages generated by the plurality of patient care user nodes; and
dynamically modifying the patient care node group based on one or more of the following:
a change in patient care scope;
a change in patient location;
a change in patient care personnel; and/or
patient data obtained from voice messages sent by the plurality of patient care user nodes.

29. The non-transitory computer readable storage medium of claim 28 further configured to store computer instructions that when executed cause one or more processors to generate a log of patient care user node messages and patient care node group modifications.

30. The non-transitory computer readable storage medium of claim 28 wherein a first patient care user node of the plurality of patient care user nodes comprises a two-device node comprising an intermediate communication device paired with a voice unit, wherein the intermediate communication device is connected to a group communication network.

31. The non-transitory computer readable storage medium of claim 30 wherein the intermediate communication device is paired to the voice unit using Bluetooth LE.

32. The non-transitory computer readable storage medium of claim 28 wherein a first patient care user node of the plurality of patient care user nodes comprises a stand-alone device connected to a group communication network.

33. The non-transitory computer readable storage medium of claim 28 further configured to store computer instructions that when executed cause one or more processors to perform a medication conflict check based on patient data obtained from voice messages sent by the plurality of patient care user nodes.

34. The non-transitory computer readable storage medium of claim 28 wherein each patient care bot node is configured to pose questions to a patient care user node user to lead the user through patient-related process, a pre-scripted protocol, and/or a data collection process.

35. A method of managing a communications group, the method comprising:
receiving patient identification data;
creating a patient care record based on the received patient identification data;
pairing the patient care record with a patient care node group, wherein the patient care node group comprises a plurality of patient care user nodes and a patient care bot node;
collecting patient data via voice messages generated by the plurality of patient care user nodes; and
detecting a change in one of the following based on the collected patient data;
patient care scope;
patient location; and/or
patient care personnel;
dynamically modifying the patient care node group based on the detected change.

36. The method of claim 35 further comprising sending a patient care notification based on collected patient care data to one of the following:
a pharmacy;
patient care food service;
surgery scheduling;
insurance.

37. The method of claim 35 wherein at least one patient care user node of plurality of patient care user nodes comprises a two-device node comprising an intermediate communication device paired with a voice unit.

38. The method of claim 37 wherein the intermediate communication device is connected to a group communication network.

39. The method of claim 38 wherein intermediate communication device is paired to the voice unit using a wireless communication protocol.

40. The method of claim 35 wherein at least one patient care user node of plurality of patient care user nodes comprises a stand-alone device connected to a group communication network.

41. A method of managing a communications group, the method comprising:
receiving an event identification message identifying an event requiring emergency response assistance;
creating a response node group comprising a plurality of response node group communication nodes, wherein the plurality of response node group communication nodes comprises a first response node group user node and a risk identification node, wherein the risk identification node comprises a risk identification bot node and/or a risk identification user node, wherein any bot node comprises a software application configured to perform one or more tasks over the Internet;
evaluating response node group user node voice message content;
identifying a risk based on the evaluated response node group user node voice message content;
modifying the response node group, wherein modifying the response node group comprises adding a risk assessment node to the response node group, wherein the risk assessment node comprises a risk assessment bot node and/or a risk assessment user node; and
providing information and/or recommended actions to the first response team user node.

42. The method of claim 41 wherein the first response node group user node comprises a two-device node comprising an intermediate communication device paired with a voice unit, wherein the intermediate communication device is connected to a group communication network.

43. The method of claim 42 wherein the intermediate communication device is paired to the voice unit using Bluetooth LE.

44. The method of claim 41 wherein the first response node group user node comprises a stand-alone device connected to a group communication network.

45. The method of claim 41 wherein evaluating response node group user node voice message content comprises converting voice message audio data to text data and evaluating text data content.

46. The method of claim 41 wherein the risk assessment node comprises a risk assessment bot node and further wherein the risk assessment bot node is accessed at or obtained from a bot library.

47. The method of claim 46 wherein the risk assessment bot node is configured to pose questions to one or more response node group user nodes to request additional information to assist in evaluating the identified risk and/or response node group user node voice message audio content.

48. The method of claim 41 further comprising dynamically modifying user node membership of the response node group based on risk assessment information collected from the response node group member nodes.

49. A non-transitory computer readable storage medium configured to store computer instructions that when executed cause one or more processors to perform:
receiving an event identification message identifying an event requiring emergency response assistance;
creating a response node group comprising a plurality of response node group communication nodes, wherein the plurality of response node group communication nodes comprises a first response node group user node, a second response node group user node, and a risk identification bot node, wherein the risk identification bot node comprises a software application configured to perform one or more tasks over the Internet; and
adding a risk assessment bot node to the response node group, wherein the risk assessment bot node comprises a software application configured to perform one or more tasks over the Internet; and
wherein the risk identification node is configured to evaluate response node group user node voice message content and to identify a risk based on the evaluated response node group user node voice message content;
further wherein the risk assessment node is selected based on a risk identified by the risk identification bot node and is configured to provide information and/or recommended actions to the first and second response team user nodes based on evaluated response node group user node voice message content.

50. The non-transitory computer readable storage medium of claim 49 wherein at least one of the first and second response node group user nodes comprises a two-device node comprising an intermediate communication device paired with a voice unit, wherein the intermediate communication device is connected to a group communication network.

51. The non-transitory computer readable storage medium of claim 50 wherein the intermediate communication device is paired to the voice unit using Bluetooth LE.

52. The non-transitory computer readable storage medium of claim 49 wherein at least one of the first and second response node group user nodes comprises a stand-alone device connected to a group communication network.

53. The non-transitory computer readable storage medium of claim 49 wherein the risk identification node evaluating response node group user node voice message content comprises converting voice message audio data to text data and evaluating text data content.

54. The non-transitory computer readable storage medium of claim 49 wherein the risk assessment bot node is accessed at or obtained from a bot library.

55. The non-transitory computer readable storage medium of claim 49 further comprising the group management system dynamically modifying user node membership of the response node group based on risk assessment information collected from the response node group member nodes.

56. A method of managing a communications group, the method comprising:
a group management system receiving an event identification message identifying an event requiring emergency response assistance;
the group management system creating a response node group comprising a first response node group user node, a second response node group user node, and a risk identification bot node, wherein the risk identification bot node comprises a risk identification software application configured to perform one or more tasks over the Internet, wherein the risk identification bot node is configured to evaluate response node group user node voice message audio content;
identifying a risk based on the evaluated response node group user node voice message audio content;
adding a risk assessment bot node to the response node group, wherein the risk assessment bot node is configured to assist in evaluating a risk identified by the risk identification bot node; and
the risk assessment bot node providing information and/or recommended actions to the first and second response team user nodes based on evaluation of response node group user node voice message audio content.

57. The method of claim 56 wherein at least one of the first and second response node group user nodes comprises a two-device node comprising an intermediate communication device paired with a voice unit, wherein the intermediate communication device is connected to a group communication network and further wherein the intermediate communication device is paired to the voice unit using a wireless communication protocol.

58. The method of claim 57 wherein at least one of the first and second response node group user nodes comprises a stand-alone device connected to a group communication network.

59. The method of claim 56 wherein the risk assessment bot node is configured to pose questions to response node group user nodes to request additional information based on based on evaluation of response node group user node voice message audio content.
